(19) United States
(12) Patent Application Publication
(10) Pub. No.: US 2023/0331840 A1
(43) Pub. Date: Oct. 19, 2023

(54) CD22 ANTIBODY AND APPLICATION THEREOF

(71) Applicant: FAPON BIOTHERAPY INC., Shenzhen, Guangdong (CN)

(72) Inventors: Xiaolong DU, Shenzhen, Guangdong (CN); Liang PENG, Shenzhen, Guangdong (CN); Baolei WANG, Shenzhen, Guangdong (CN); Chunxi GONG, Shenzhen, Guangdong (CN); Ying LUO, Shenzhen, Guangdong (CN); Lijun YE, Shenzhen, Guangdong (CN); Xianjin WANG, Shenzhen, Guangdong (CN)

(21) Appl. No.: 18/023,389

(22) PCT Filed: Aug. 23, 2021
(86) PCT No.: PCT/CN2021/114103

§ 371 Date: Feb. 27, 2023

(30) Foreign Application Priority Data
Aug. 27, 2020 (CN) 202010878339.7

(51) Int. Cl. C07K 16/28; C07K 14/705; C07K 14/725

(52) U.S. Cl. CPC C07K 16/2803; C07K 14/70517; C07K 14/7051; C07K 2317/565; C07K 2317/567; C07K 2317/622; C07K 2317/52; C07K 2319/33; C07K 2319/03; A61K 38/00

(57) ABSTRACT

Provided is an antibody or antigen-binding fragment thereof that can specifically recognize CD22. The antibody contains a CDR sequence selected from at least one of the following or an amino acid sequence at least 95% identical thereto: a heavy-chain variable region CDR sequence: SEQ ID NOs: 1-15, and a light-chain variable region CDR sequence as shown in SEQ IN NOs: 16-30. The antibody can specifically recognize CD22 and has high affinity to CD22.

### PRIORITY INFORMATION

The present application claims the priority and benefit of patent application with Patent Application No. 202010878339.7 field to the China National Intellectual Property Administration on Aug. 27, 2020, the entire content of which is incorporated herein by reference.

### SEQUENCE LISTING

The present application contains a Sequence Listing which has been submitted electronically in ASCII format and is hereby incorporated by reference in its entirety. Said ASCII copy is named_Sequence_Listing.txt and is 131,903 bytes in size, and the Sequence listing is identical to the international application No. PCT/CN2021/114103 filed on Aug. 23, 2021, except the following formal amendments: 1) for identifier<130>, PN202108FPSW has been added; 2) identifier <150> and <151>, CN202010878339.7 and 2020-08-27 have been added; 3) for identifiers<220> and <223>, the comments for artificial sequences have been added; 4) The sequences with less than 4 amino acids is supplemented with Xaa for 4 amino acids, and Xaa is not exist, just for completing the sequence listing.

### TECHNICAL FIELD

The present disclosure relates to the field of biotechnology, in particular to a CD22 antibody and use thereof. More specifically, the present disclosure relates to an antibody or an antigen-binding fragment thereof that can specifically recognize CD22, a nucleic acid molecule, an expression vector, a recombinant cell, a chimeric antigen receptor, a CART cell, a pharmaceutical composition, a pharmaceutical use, as well as a kit for detecting CD22.

### BACKGROUND

Hematological malignancy is one of the top ten high incidence of malignancies in China, and ranks sixth in the incidence of tumors. Especially, acute lymphoblastic leukemia (ALL), which mostly occurs in adolescents, is the malignancy with the highest morbidity and mortality among people under 35 years old, with acute B-lymphoblastic leukemia (B-ALL) being the most common.

CD22, a type I transmembrane glycoprotein, is a member of the sialic acid binding immunoglobulin-like lectin family. As an inhibitory co-receptor of B cell receptors (BCRs), CD22 negatively regulates B cell activation signals. CD22 can specifically bind to a glycoprotein ligand containing α-2, 6-linked sialic acid, the antigen activates BCR, which also rapidly phosphorylates tyrosine in the immunoreceptor tyrosine-based inhibitory motif in the cytoplasmic region of CD22, and activates downstream signal molecules to inhibit calcium ion influx, thus weakening the BCR signals. CD22 participates in the homing process of B cells. Since CD22 is relatively specifically expressed on the surface of B cells, it has become a good target for regulating B cell immunity and treating some B cell tumors.

CD22 has a molecular weight of 140 kDa. The extracellular domain of CD22 contains seven Ig domains (residues 20-687M). The V-set Ig domain at the farthest end primarily functions to bind a 2,6-sialic acid (α 2,6-sia) ligand, and the linked C2-set Ig domain may function to allow the correct folding of V-set Ig domain. The intracellular domain of CD22 includes immunoreceptor tyrosine-based inhibitory motif (ITIM) and immunoreceptor tyrosine-based activatory motif (ITAM). Ig-like domains 1 and 2 contain ligand binding regions, and when one or more of the six conservative tyrosine residues are phosphorylated, various effector molecules are recruited into the cytoplasmic domain. CD22 α (647aa) and CD22 β (847aa) are two subtypes of CD22, their extracellular domains have 5 and 7 Ig domains, respectively. These two cDNA subtypes originate from different splicing of the same genes.

Studies have shown that approximately 90% of R/R B-ALL patients achieve complete remission (CR) after receiving CART19. Although the initial response rate is very high, many patients have relapses. More than 30% of relapsed patients treated with Blinatumomab and more than 60% of relapsed patients who have used CAR-T19 show loss of the CD19 antigen targets, which makes CD19 specific immunotherapy unable to identify malignant cells. These phenomena also explain the advantages and disadvantages of antigen specific immunotherapies. CD22 is expressed in B-ALL tumors and lymphoid initiating cells, as well as in CD19 negative recurrent cells after CART19 therapy. Therefore, CD22 has a good application prospect both as a therapeutic target for treating B-cell leukemia alone and as an auxiliary target for CD19, and due to the low preparation cost of CART cells as well, it can be used in the treatment of patients immediately.

In view of the above, there is an urgent need to disclose a universal CART cell targeting CD22.

### SUMMARY

The present disclosure aims to solve at least one of the technical problems in the related technologies to a certain extent. To this end, the present disclosure provides a specific antibody against CD22 and a universal CART cell targeting CD22.

In a first aspect of the present disclosure, there is provided an antibody or antigen-binding fragment thereof that can specifically recognize CD22. According to the embodiments of the present disclosure, the antibody contains a CDR sequence selected from at least one of the following or an amino acid sequence having at least 95% identity thereto: heavy-chain variable region CDR sequences as shown in SEQ ID NOs: 1-15, and light-chain variable region CDR sequences as shown in SEQ IN NOs: 16-30. The antibody according to the embodiments of the present disclosure can specifically recognize CD22 and has high affinity to CD22.

(SEQ ID NO: 1)
GYSITSGYY.
(SEQ ID NO: 2)
ISYDGSN.
(SEQ ID NO: 3)
TK.
(SEQ ID NO: 4)
GYNFTSYW.
(SEQ ID NO: 5)
IYPGSGNT.
(SEQ ID NO: 6)
AR.
(SEQ ID NO: 7)
GYTFSSYW.
(SEQ ID NO: 8)
ILPGSGST.
(SEQ ID NO: 9)
AR.
(SEQ ID NO: 10)
GYTFTDSI.
(SEQ ID NO: 11)
FYPGSGSI.
(SEQ ID NO: 12)
ARHE.
(SEQ ID NO: 13)
GYTFSSYW.
(SEQ ID NO: 14)
IYPSDSYT.
(SEQ ID NO: 15)
TR.
(SEQ ID NO: 16)
GNIHNY.
(SEQ ID NO: 17)
NAK.
(SEQ ID NO: 18)
QHFWSTP.
(SEQ ID NO: 19)
GNIHNY.
(SEQ ID NO: 20)
NAK.
(SEQ ID NO: 21)
QHFWSTP.
(SEQ ID NO: 22)
ENIYSY.
(SEQ ID NO: 23)
NAK.
(SEQ ID NO: 24)
QHHYGSP.
(SEQ ID NO: 25)
SSVNY.
(SEQ ID NO: 26)
YTS.
(SEQ ID NO: 27)
QQFTSSP.
(SEQ ID NO: 28)
KTISKY.
(SEQ ID NO: 29)
SGS.
(SEQ ID NO: 30)
QQHNEYPW.

According to the embodiments of the present disclosure, the above antibody or antigen-binding fragment thereof can further include at least one of the following additional technical features:

According to the embodiments of the present disclosure, the antibody includes: heavy-chain variable region sequences CDR1, CDR2 and CDR3 as shown in SEQ ID NOs: 1, 2 and 3, or amino acid sequence having at least 95% identity to SEQ ID NOs: 1, 2 and 3, respectively; or heavy-chain variable region sequences CDR1, CDR2 and CDR3 as shown in SEQ ID NOs: 4, 5 and 6 or amino acid sequence having at least 95% identity to SEQ ID NOs: 4, 5 and 6, respectively; or heavy-chain variable region sequences CDR1, CDR2 and CDR3 as shown in SEQ ID NOs: 7, 8 and 9 or amino acid sequence having at least 95% identity to SEQ ID NOs: 7, 8 and 9, respectively; or heavy-chain variable region sequences CDR1, CDR2 and CDR3 as shown in SEQ ID NOs: 10, 11 and 12 or amino acid sequence having at least 95% identity to SEQ ID NOs: 10, 11 and 12, respectively; or heavy-chain variable region sequences CDR1, CDR2 and CDR3 as shown in SEQ ID NOs: 13, 14 and 15 or amino acid sequence having at least 95% identity to SEQ ID NOs: 13, 14 and 15, respectively.

According to the embodiments of the present disclosure, the antibody includes: light-chain variable region sequences CDR1, CDR2 and CDR3 as shown in SEQ ID NOs: 16, 17 and 18 or amino acid sequence having at least 95% identity to SEQ ID NOs: 16, 17 and 18, respectively; or light-chain variable region sequences CDR1, CDR2 and CDR3 as shown in SEQ ID NOs: 19, 20 and 21 or amino acid sequence having at least 95% identity to SEQ ID NOs: 19, 20 and 21, respectively; or light-chain variable region sequences CDR1, CDR2 and CDR3 as shown in SEQ ID NOs: 22, 23 and 24 or amino acid sequence having at least 95% identity to SEQ ID NOs: 22, 23 and 24, respectively; or light-chain variable region sequences CDR1, CDR2 and CDR3 as shown in SEQ ID NOs: 25, 26 and 27 or amino acid sequence having at least 95% identity to SEQ ID NOs: 25, 26 and 27, respectively; or light-chain variable region sequences CDR1, CDR2 and CDR3 as shown in SEQ ID NOs: 28, 29 and 30 or amino acid sequence having at least 95% identity to SEQ ID NOs: 28, 29 and 30, respectively.

According to the embodiments of the present disclosure, the antibody includes: heavy-chain variable region sequences CDR1, CDR2 and CDR3 as shown in SEQ ID NOs: 1, 2 and 3, or amino acid sequence having at least 95% identity to SEQ ID NOs: 1, 2 and 3, respectively, and light-chain variable region sequences CDR1, CDR2 and CDR3 as shown in SEQ ID NOs: 16, 17 and 18 or amino acid sequence having at least 95% identity to SEQ ID NOs: 16, 17 and 18, respectively; or heavy-chain variable region sequences CDR1, CDR2 and CDR3 as shown in SEQ ID NOs:4, 5 and 6, or amino acid sequence having at least 95% identity to SEQ ID NOs: 4, 5 and 6, respectively, and light-chain variable region sequences CDR1, CDR2 and CDR3 as shown in SEQ ID NOs: 19, 20 and 21 or amino acid sequence having at least 95% identity to SEQ ID NOs: 19, 20 and 21, respectively; or heavy-chain variable region sequences CDR1, CDR2 and CDR3 as shown in SEQ ID NOs:7, 8 and 9, or amino acid sequence having at least 95% identity to SEQ ID NOs: 7, 8 and 9, respectively, and light-chain variable region sequences CDR1, CDR2 and CDR3 as shown in SEQ ID NOs: 22, 23 and 24 or amino acid sequence having at least 95% identity to SEQ ID NOs: 22, 23 and 24, respectively; or heavy-chain variable region sequences CDR1, CDR2 and CDR3 as shown in SEQ ID NOs:10, 11 and 12, or amino acid sequence having at least 95% identity to SEQ ID NOs: 10, 11 and 12, respectively, and light-chain variable region sequences CDR1, CDR2 and CDR3 as shown in SEQ ID NOs: 25, 26 and 27 or amino acid sequence having at least 95% identity to SEQ ID NOs: 25, 26 and 27, respectively; or heavy-chain variable region sequences CDR1, CDR2 and CDR3 as shown in SEQ ID NOs:13, 14 and 15, or amino acid sequence having at least 95% identity to SEQ ID NOs: 13, 14 and 15, respectively, and light-chain variable region sequences CDR1, CDR2 and CDR3 as shown in SEQ ID NOs: 28, 29 and 30 or amino acid sequence having at least 95% identity to SEQ ID NOs: 28, 29 and 30, respectively.

According to the embodiments of the present disclosure, the antibody or antigen-binding fragment thereof specifically recognizes the extracellular region of CD22.

According to the embodiments of the present disclosure, the antibody contains at least one of a heavy-chain framework region sequence and a light-chain framework region sequence. According to the embodiments of the present disclosure, at least one part of at least one of the heavy chain framework region sequence and the light chain framework region sequence is derived from at least one of a mouse-derived antibody, a human-derived antibody, a primate-derived antibody or a mutant thereof.

According to the embodiments of the present disclosure, the antibody has a heavy chain variable region having the amino acid sequence as shown in any one of SEQ ID NOs: 31-35.

(SEQ ID NO: 31)
DVQLQESGPGLVKPSQSLSLTCSVTGYSITSGYYWNWIRQ
FPGNKLEWMGYISYDGSNNYNPSLKNRISITRDTSKNQFF
LKLNSVTTEDTATYYCTKGGYGYYFDYWGQGTTLTVSS.
(SEQ ID NO: 32)
QVQLQQPGAELVKPGTSVKLSCKASGYNFTSYWINWWKLR
PGQGLEWIGDIYPGSGNTNYNEKFKSKATLTVDTSSTTAY
MQLSSLASEDSALYYCARRGYLDYWGQGTTLTVSS.
(SEQ ID NO: 33)
QVQLQQSGAELMKPGASVKISCKATGYTFSSYWIEWWKQR
PGHGLEWIGEILPGSGSTNYNEKFKGKATFTADTSSNTAY
MQLSSLTSEDSAVYYCARWGQLGLFYAMDYWGQGTSVTVS
S.
(SEQ ID NO: 34)
KVQLQQSGAGLVKPGASVKLSCKASGYTFTDSILHWLMQR
SGQGLEWIGWFYPGSGSIKYNEKFKDKATLTADKSSSTVY
MELSRLTSEDSAFYFCARHEDGYDGFAYWGQGTLVTVSA.
(SEQ ID NO: 35)
QVQLQQPGAELVRPGASVKLSCKASGYTFSSYWINWWKQR
PGQGLEWIGNIYPSDSYTNYNQKFKDKATLTVDKSSSTAY
MQLSSPTSEDSAVYYCTREGHYYGSFGAMDYWGQGTSVTV
SS.
According to the embodiments of the present
disclosure, the antibody has a light chain
variable region havingthe amino acid sequence
as shown in any one of SEQ ID NOS: 36-40.
(SEQ ID NO: 36)
DIQMTQSPASLSASVGETVTITCRASGNIHNYLAWYQQKQ
GKSPQLLVYNAKTLADGVPSRFSGSGSGTQYSLKINSLQP
EDFGSYYCQHFWSTPPTFGGGTKLEIKR.
(SEQ ID NO: 37)
DIQMTQSPASLSASVGETVTITCRASGNIHNYLAWYQQKQ
GKSPQLLVYNAKTLADGVPSRFSGSGSGTQYSLTINSLQP
EDFGSYYCQHFWSTPLTFGAGTKLELKR.
(SEQ ID NO: 38)
DIQMTQSPASLSASVGETVTITCRASENIYSYLAWYQQKQ
GKSPQLLVYNAKTLAEGVPSRFSGSGSGTQFSLKINSLQP
EDFGSYYCQHHYGSPLTFGAGTKLELKR.
(SEQ ID NO: 39)
ENVLTQSPAIMSASLGEKVTMSCRASSSVNYIFWYQQKSD
ASPKLWIYYTSNLAPGVPARFSGSGSGNSYSLTISSMEGE
DAATYYCQQFTSSPFTFGSGTKLEIKR.
(SEQ ID NO: 40)
DVQITQSPSYLAASPGETITINCRASKTISKYLAWYQEKP
GKTNKLLIYSGSTLQSGIPSRFSGSGSGTDFTLTISSLEP
EDFAMYYCQQHNEYPWTFGGGTKLEIKR.

According to the embodiments of the present disclosure, the antibody contains at least one of a heavy-chain constant region and a light-chain constant region, and at least one part of at least one of the heavy chain constant region and the light chain constant region is derived from at least one of a mouse-derived antibody, a human-derived antibody, a primate-derived antibody or a mutant thereof.

According to the embodiments of the present disclosure, the light chain constant region and heavy chain constant region of the antibody are both derived from human-derived IgG antibody or a mutant thereof.

According to the embodiments of the present disclosure, the light chain constant region and heavy chain constant region of the antibody are both derived from human-derived IgG 1, 2 or 4. Furthermore, the immunogenicity of the antibody can be effectively reduced.

Wherein, in this application, the above SEQ ID NOs: 31 and 36 represent the heavy chain variable region and light chain variable region of antibody FC2-117, the above SEQ ID NOs: 32 and 37 represent the heavy chain variable region and light chain variable region of antibody FC2-070, the above SEQ ID NOs: 33 and 38 represent the heavy chain variable region and light chain variable region of antibody FC2-153, the above SEQ ID NOs: 34 and 39 represent the heavy chain variable region and light chain variable region of antibody FC2-201, and the above SEQ ID NOs: 35 and 40 represent the heavy chain variable region and light chain variable region of antibody FC2-203.

According to the embodiments of the present disclosure, the antibody is a single chain antibody, a polymer antibody, a CDR grafted antibody or a micromolecular antibody.

According to the embodiments of the present disclosure, the antibody is a single chain antibody.

According to the embodiments of the present disclosure, the antibody has the amino acid sequence as shown in SEQ ID NOs:41-50.

(SEQ ID NO: 41)
SGSGSGTQYSLKINSLQPEDFGSYYCQHFWSTPPTFGGGT
KLEIKRGGGGSGGGGSGGGGSDVQLQESGPGLVKPSQSLS
LTCSVTGYSITSGYYWNWIRQFPGNKLEWMGYISYDGSNN
YNPSLKNRISITRDTSKNQFFLKLNSVTTEDTATYYCTKG
GYGYYFDYWGQGTTLTVSS
(SEQ ID NO: 42)
DIQMTQSPASLSASVGETVTITCRASGNIHNYLAWYQQKQ
GKSPQLLVYNAKTLADGVPSRFSGSGSGTQYSLTINSLQP
EDFGSYYCQHFWSTPLTFGAGTKLELKRGGGGSGGGGSGG
GGSQVQLQQPGAELVKPGTSVKLSCKASGYNFTSYWINWW
KLRPGQGLEWIGDIYPGSGNTNYNEKFKSKATLTVDTSST
TAYMQLSSLASEDSALYYCARRGYLDYWGQGTTLTVSS
(SEQ ID NO: 43)
DIQMTQSPASLSASVGETVTITCRASENIYSYLAWYQQKQ
GKSPQLLVYNAKTLAEGVPSRFSGSGSGTQFSLKINSLQP
EDFGSYYCQHHYGSPLTFGAGTKLELKRGGGGSGGGGSGG
GGSQVQLQQSGAELMKPGASVKISCKATGYTFSSYWIEWW
KQRPGHGLEWIGEILPGSGSTNYNEKFKGKATFTADTSSN
TAYMQLSSLTSEDSAVYYCARWGQLGLFYAMDYWGQGTSV
TVSS
(SEQ ID NO: 44)
ENVLTQSPAIMSASLGEKVTMSCRASSSVNYIFWYQQKSD
ASPKLWIYYTSNLAPGVPARFSGSGSGNSYSLTISSMEGE
DAATYYCQQFTSSPFTFGSGTKLEIKRGGGGSGGGGSGGG
GSKVQLQQSGAGLVKPGASVKLSCKASGYTFTDSILHWLM
QRSGQGLEWIGWFYPGSGSIKYNEKFKDKATLTADKSSST
VYMELSRLTSEDSAFYFCARHEDGYDGFAYWGQGTLVTVS
A
(SEQ ID NO: 45)
DVQITQSPSYLAASPGETITINCRASKTISKYLAWYQEKP
GKTNKLLIYSGSTLQSGIPSRFSGSGSGTDFTLTISSLEP
EDFAMYYCQQHNEYPWTFGGGTKLEIKRGGGGSGGGGSGG
GGSQVQLQQPGAELVRPGASVKLSCKASGYTFSSYWINWW
KQRPGQGLEWIGNIYPSDSYTNYNQKFKDKATLTVDKSSS
TAYMQLSSPTSEDSAVYYCTREGHYYGSFGAMDYWGQGTS
VTVSS
(SEQ ID NO: 46)
DVQLQESGPGLVKPSQSLSLTCSVTGYSITSGYYWNWIRQ
FPGNKLEWMGYISYDGSNNYNPSLKNRISITRDTSKNQFF
LKLNSVTTEDTATYYCTKGGYGYYFDYWGQGTTLTVSSGG
GGSGGGGSGGGGSDIQMTQSPASLSASVGETVTITCRASG
NIHNYLAWYQQKQGKSPQLLVYNAKTLADGVPSRFSGSGS
GTQYSLKINSLQPEDFGSYYCQHFWSTPPTFGGGTKLEIK
R
(SEQ ID NO: 47)
QVQLQQPGAELVKPGTSVKLSCKASGYNFTSYWINWWKLR
PGQGLEWIGDIYPGSGNTNYNEKFKSKATLTVDTSSTTAY
MQLSSLASEDSALYYCARRGYLDYWGQGTTLTVSSGGGGS
GGGGSGGGGSDIQMTQSPASLSASVGETVTITCRASGNIH
NYLAWYQQKQGKSPQLLVYNAKTLADGVPSRFSGSGSGTQ
YSLTINSLQPEDFGSYYCQHFWSTPLTFGAGTKLELKR
(SEQ ID NO: 48)
QVQLQQSGAELMKPGASVKISCKATGYTFSSYWIEWWKQR
PGHGLEWIGEILPGSGSTNYNEKFKGKATFTADTSSNTAY
MQLSSLTSEDSAVYYCARWGQLGLFYAMDYWGQGTSVTVS
SGGGGSGGGGSGGGGSDIQMTQSPASLSASVGETVTITCR
ASENIYSYLAWYQQKQGKSPQLLVYNAKTLAEGVPSRFSG
SGSGTQFSLKINSLQPEDFGSYYCQHHYGSPLTFGAGTKL
ELKR
(SEQ ID NO: 49)
KVQLQQSGAGLVKPGASVKLSCKASGYTFTDSILHWLMQR
SGQGLEWIGWFYPGSGSIKYNEKFKDKATLTADKSSSTVY
MELSRLTSEDSAFYFCARHEDGYDGFAYWGQGTLVTVSAG
GGGSGGGGSGGGGSENVLTQSPAIMSASLGEKVTMSCRAS
SSVNYIFWYQQKSDASPKLWIYYTSNLAPGVPARFSGSGS
GNSYSLTISSMEGEDAATYYCQQFTSSPFTFGSGTKLEIK
R
(SEQ ID NO: 50)
QVQLQQPGAELVRPGASVKLSCKASGYTFSSYWINWWKQR
PGQGLEWIGNIYPSDSYTNYNQKFKDKATLTVDKSSSTAY
MQLSSPTSEDSAVYYCTREGHYYGSFGAMDYWGQGTSVTV
SSGGGGSGGGGSGGGGSDVQITQSPSYLAASPGETITINC
RASKTISKYLAWYQEKPGKTNKLLIYSGSTLQSGIPSRFS
GSGSGTDFTLTISSLEPEDFAMYYCQQHNEYPWTFGGGTK
LEIKR

Wherein, in this application, the antibody having the amino acid sequence as shown in the above SEQ ID NO: 41 or 46 is called as FC2-117 single chain antibody, and the antibody having the amino acid sequence as shown in the above SEQ ID NO: 42 or 47 is called as FC2-070 single chain antibody, the antibody having the amino acid sequence as shown in the above SEQ ID NO: 43 or 48 is called as FC2-153 single chain antibody, the antibody having the amino acid sequence as shown in the above SEQ ID NO: 44 or 49 is called as FC2-201 single chain antibody, and the antibody having the amino acid sequence as shown in the above SEQ ID NOs: 45 and 50 is called as FC2-203 single chain antibody. Wherein, the antibody having the amino acid sequence as shown in SEQ ID NOs: 41-45 can be expressed as VL-Link-VH (VL represents the light chain variable region, VH represents the heavy chain variable region, and Link represents the link chain connecting VL and VH) from the N-end to the C-end, and the antibody having the amino acid sequence as shown in SEQ ID NOs: 46-50 can be expressed as VH-Link-VL (VL represents the light chain variable region, VH represents the heavy chain variable region, and Link represents the link chain connecting VL and VH).

According to the embodiments of the present disclosure, the micromolecular antibody includes at least one of Fab antibody, Fv antibody, single domain antibody and minimum recognition unit.

In the second aspect of the present disclosure, there is provided a nucleic acid. According to the embodiments of the present disclosure, the nucleic acid molecule encodes the antibody or antigen-binding fragment thereof described above. The antibody or antigen-binding fragment thereof encoded by the nucleic acid molecule according to the embodiments of the present disclosure can specifically targeted to CD22 with high affinity.

According to the embodiments of the present disclosure, the above nucleic acid can further include at least one of the following additional technical features:

According to the embodiments of the present disclosure, the nucleic acid molecule is DNA.

According to the embodiments of the present disclosure, the nucleic acid molecule has a nucleotide sequence as shown in any one of SEQ ID NOs: 51-55 or has a nucleotide sequence as shown in any one of SEQ ID NOs: 56-60 or has a nucleotide sequence as shown in any one of SEQ ID NOs: 61-70.

(SEQ ID NO: 51)
GATGTACAGCTTCAGGAGTCAGGACCTGGCCTCGTGAAAC
CTTCTCAGTCTCTGTCTCTCACCTGCTCTGTCACTGGCTA
CTCCATCACCAGTGGTTATTACTGGAACTGGATCCGGCAG
TTTCCAGGAAACAAACTGGAATGGATGGGCTACATAAGCT
ACGACGGTAGCAATAACTACAACCCATCTCTCAAAAATCG
AATCTCCATCACTCGTGACACATCTAAGAACCAGTTTTTC
CTGAAGTTGAATTCTGTGACTACTGAGGACACAGCTACAT
ATTACTGTACAAAAGGGGGCTACGGCTACTACTTTGACTA
CTGGGGCCAAGGCACCACTCTCACAGTCTCCTCA
(SEQ ID NO: 52)
CAGGTCCAGCTGCAGCAGCCTGGGGCTGAGCTTGTGAAGC
CTGGGACTTCAGTGAAGCTGTCCTGCAAGGCTTCTGGCTA
CAACTTCACCAGCTACTGGATAAACTGGGTGAAGCTGAGG
CCTGGACAAGGCCTTGAGTGGATTGGAGATATTTATCCTG
GTAGTGGTAATACTAATTACAATGAGAAGTTCAAGAGCAA
GGCCACACTGACTGTAGACACATCCTCCACCACAGCCTAC
ATGCAACTTAGTAGCCTGGCCTCTGAGGACTCTGCTCTCT
ATTACTGTGCAAGACGGGGGTATCTTGACTACTGGGGCCA
AGGCACCACTCTCACAGTCTCCTCA
(SEQ ID NO: 53)
CAGGTTCAGCTGCAGCAGTCTGGAGCTGAGCTGATGAAGC
CTGGGGCCTCAGTGAAGATATCCTGCAAGGCTACTGGCTA
CACATTCAGTAGCTACTGGATAGAGTGGGTAAAGCAGAGG
CCTGGACATGGCCTTGAGTGGATTGGAGAGATTTTACCTG
GAAGTGGTAGTACTAACTACAATGAGAAGTTCAAGGGCAA
GGCCACATTCACTGCAGATACATCCTCCAACACAGCCTAC
ATGCAACTCAGCAGCCTGACATCTGAGGACTCTGCCGTCT
ATTACTGTGCAAGATGGGGGCAGCTCGGGCTTTTTTATGC
TATGGACTACTGGGGTCAAGGAACCTCAGTCACCGTCTCC
TCA
(SEQ ID NO: 54)
AAGGTCCAGCTGCAGCAGTCTGGAGCTGGGCTGGTGAAAC
CCGGGGCATCAGTGAAGCTGTCCTGCAAGGCTTCTGGCTA
CACCTTCACTGACTCTATTTTACACTGGCTAATGCAGAGA
TCTGGACAGGGTCTTGAGTGGATTGGGTGGTTTTACCCTG
GAAGTGGTAGTATAAAGTACAATGAGAAATTCAAGGACAA
GGCCACATTGACTGCGGACAAGTCCTCCAGCACAGTCTAT
ATGGAGCTTAGTAGATTGACATCTGAAGACTCTGCGTTCT
ATTTCTGTGCAAGGCACGAAGATGGTTACGACGGGTTTGC
TTACTGGGGCCAAGGGACTCTGGTCACTGTCTCTGCA
(SEQ ID NO: 55)
CAGGTCCAACTGCAGCAGCCTGGGGCTGAGCTGGTGAGGC
CTGGGGCTTCAGTGAAGCTGTCCTGCAAGGCTTCTGGCTA
CACCTTCAGCAGCTACTGGATAAACTGGGTGAAGCAGAGG
CCTGGACAAGGCCTTGAGTGGATCGGAAATATTTATCCTT
CTGATAGTTATACTAACTACAATCAAAAGTTCAAGGACAA
GGCCACATTGACTGTAGACAAATCCTCCAGTACAGCCTAC
ATGCAGCTCAGCAGCCCGACATCTGAGGACTCTGCGGTCT
ATTACTGTACAAGAGAGGGGCATTACTACGGATCCTTCGG
TGCTATGGACTACTGGGGTCAAGGAACCTCAGTCACCGTC
TCCTCA
(SEQ ID NO: 56)
GACATCCAGATGACTCAGTCTCCAGCCTCCCTATCTGCAT
CTGTGGGAGAAACTGTCACCATCACATGTCGAGCAAGTGG
GAATATTCACAATTATTTAGCATGGTATCAGCAGAAACAG
GGAAAATCTCCTCAGCTCCTGGTCTATAATGCAAAAACCT
TAGCAGATGGTGTGCCATCAAGGTTCAGTGGCAGTGGATC
AGGAACACAATATTCTCTCAAGATCAACAGCCTGCAGCCT
GAAGATTTTGGGAGTTATTACTGTCAACATTTTTGGAGTA
CTCCTCCGACGTTCGGTGGAGGCACCAAGCTGGAAATCAA
ACGG
(SEQ ID NO: 57)
GACATCCAGATGACTCAGTCTCCAGCCTCCCTATCTGCAT
CTGTGGGAGAAACTGTCACCATCACATGTCGAGCAAGTGG
GAATATTCACAATTATTTAGCATGGTATCAGCAGAAACAG
GGAAAATCTCCTCAGCTCCTGGTCTATAATGCAAAAACCT
TAGCAGATGGTGTGCCATCAAGGTTCAGTGGCAGTGGATC
AGGAACACAATATTCTCTCACGATCAACAGCCTGCAGCCT
GAAGATTTTGGGAGTTACTACTGTCAACATTTTTGGAGTA
CTCCTCTCACGTTCGGTGCTGGGACCAAGCTGGAGCTGAA
ACGG
(SEQ ID NO: 58)
GACATCCAGATGACTCAGTCTCCAGCCTCCCTATCTGCAT
CTGTGGGAGAAACTGTCACCATCACATGTCGAGCAAGTGA
GAATATTTACAGTTATTTAGCATGGTATCAGCAGAAACAG
GGAAAATCTCCTCAGCTCCTGGTCTATAATGCAAAAACCT
TAGCAGAAGGTGTGCCATCAAGGTTCAGTGGCAGTGGATC
AGGCACACAGTTTTCTCTGAAGATCAACAGCCTGCAGCCT
GAAGATTTTGGGAGTTATTACTGTCAACATCATTATGGTA
GTCCGCTCACGTTCGGTGCTGGGACCAAGCTGGAGCTGAA
ACGG
(SEQ ID NO: 59)
GAAAATGTGCTCACCCAGTCTCCAGCAATCATGTCTGCAT
CTCTAGGGGAGAAGGTCACCATGAGCTGCAGGGCCAGCTC
AAGTGTAAATTACATATTCTGGTACCAGCAGAAGTCAGAT
GCCTCCCCCAAACTATGGATTTATTACACATCCAACCTGG
CTCCTGGAGTCCCAGCTCGCTTCAGTGGCAGTGGGTCTGG
GAACTCTTATTCTCTCACAATCAGCAGCATGGAGGGTGAA
GATGCTGCCACTTATTACTGCCAGCAGTTTACTAGTTCCC
CATTCACGTTCGGCTCGGGGACAAAGTTGGAAATAAAACG
G
(SEQ ID NO: 60)
GATGTCCAGATAACCCAGTCTCCATCTTATCTTGCTGCAT
CTCCTGGAGAAACCATTACTATTAATTGCAGGGCAAGTAA
GACCATTAGCAAATATTTGGCCTGGTATCAAGAGAAACCT
GGGAAAACTAATAAGCTTCTTATCTACTCTGGATCCACTT
TGCAATCTGGAATTCCATCAAGGTTCAGTGGCAGTGGATC
TGGTACAGATTTCACTCTCACCATCAGTAGCCTGGAGCCT
GAAGATTTTGCAATGTATTACTGTCAACAACATAATGAAT
ACCCGTGGACGTTCGGTGGAGGCACCAAGCTGGAAATCAA
ACGG
(SEQ ID NO: 61)
GACATCCAGATGACTCAGTCTCCAGCCTCCCTATCTGCAT
CTGTGGGAGAAACTGTCACCATCACATGTCGAGCAAGTGG
GAATATTCACAATTATTTAGCATGGTATCAGCAGAAACAG
GGAAAATCTCCTCAGCTCCTGGTCTATAATGCAAAAACCT
TAGCAGATGGTGTGCCATCAAGGTTCAGTGGCAGTGGATC
AGGAACACAATATTCTCTCAAGATCAACAGCCTGCAGCCT
GAAGATTTTGGGAGTTATTACTGTCAACATTTTTGGAGTA
CTCCTCCGACGTTCGGTGGAGGCACCAAGCTGGAAATCAA
ACGGGGAGGAGGAGGCTCCGGCGGAGGAGGCTCTGGAGGA
GGAGGCAGCGATGTACAGCTTCAGGAGTCAGGACCTGGCC
TCGTGAAACCTTCTCAGTCTCTGTCTCTCACCTGCTCTGT
CACTGGCTACTCCATCACCAGTGGTTATTACTGGAACTGG
ATCCGGCAGTTTCCAGGAAACAAACTGGAATGGATGGGCT
ACATAAGCTACGACGGTAGCAATAACTACAACCCATCTCT
CAAAAATCGAATCTCCATCACTCGTGACACATCTAAGAAC
CAGTTTTTCCTGAAGTTGAATTCTGTGACTACTGAGGACA
CAGCTACATATTACTGTACAAAAGGGGGCTACGGCTACTA
CTTTGACTACTGGGGCCAAGGCACCACTCTCACAGTCTCC
TCA
(SEQ ID NO: 62)
GACATCCAGATGACTCAGTCTCCAGCCTCCCTATCTGCAT
CTGTGGGAGAAACTGTCACCATCACATGTCGAGCAAGTGG
GAATATTCACAATTATTTAGCATGGTATCAGCAGAAACAG
GGAAAATCTCCTCAGCTCCTGGTCTATAATGCAAAAACCT
TAGCAGATGGTGTGCCATCAAGGTTCAGTGGCAGTGGATC
AGGAACACAATATTCTCTCACGATCAACAGCCTGCAGCCT
GAAGATTTTGGGAGTTACTACTGTCAACATTTTTGGAGTA
CTCCTCTCACGTTCGGTGCTGGGACCAAGCTGGAGCTGAA
ACGGGGAGGAGGAGGCTCCGGCGGAGGAGGCTCTGGAGGA
GGAGGCAGCCAGGTCCAGCTGCAGCAGCCTGGGGCTGAGC
TTGTGAAGCCTGGGACTTCAGTGAAGCTGTCCTGCAAGGC
TTCTGGCTACAACTTCACCAGCTACTGGATAAACTGGGTG
AAGCTGAGGCCTGGACAAGGCCTTGAGTGGATTGGAGATA
TTTATCCTGGTAGTGGTAATACTAATTACAATGAGAAGTT
CAAGAGCAAGGCCACACTGACTGTAGACACATCCTCCACC
ACAGCCTACATGCAACTTAGTAGCCTGGCCTCTGAGGACT
CTGCTCTCTATTACTGTGCAAGACGGGGGTATCTTGACTA
CTGGGGCCAAGGCACCACTCTCACAGTCTCCTCA
(SEQ ID NO: 63)
GACATCCAGATGACTCAGTCTCCAGCCTCCCTATCTGCAT
CTGTGGGAGAAACTGTCACCATCACATGTCGAGCAAGTGA
GAATATTTACAGTTATTTAGCATGGTATCAGCAGAAACAG
GGAAAATCTCCTCAGCTCCTGGTCTATAATGCAAAAACCT
TAGCAGAAGGTGTGCCATCAAGGTTCAGTGGCAGTGGATC
AGGCACACAGTTTTCTCTGAAGATCAACAGCCTGCAGCCT
GAAGATTTTGGGAGTTATTACTGTCAACATCATTATGGTA
GTCCGCTCACGTTCGGTGCTGGGACCAAGCTGGAGCTGAA
ACGGGGAGGAGGAGGCTCCGGCGGAGGAGGCTCTGGAGGA
GGAGGCAGCCAGGTTCAGCTGCAGCAGTCTGGAGCTGAGC
TGATGAAGCCTGGGGCCTCAGTGAAGATATCCTGCAAGGC
TACTGGCTACACATTCAGTAGCTACTGGATAGAGTGGGTA
AAGCAGAGGCCTGGACATGGCCTTGAGTGGATTGGAGAGA
TTTTACCTGGAAGTGGTAGTACTAACTACAATGAGAAGTT
CAAGGGCAAGGCCACATTCACTGCAGATACATCCTCCAAC
ACAGCCTACATGCAACTCAGCAGCCTGACATCTGAGGACT
CTGCCGTCTATTACTGTGCAAGATGGGGGCAGCTCGGGCT
TTTTTATGCTATGGACTACTGGGGTCAAGGAACCTCAGTC
ACCGTCTCCTCA
(SEQ ID NO: 64)
GAAAATGTGCTCACCCAGTCTCCAGCAATCATGTCTGCAT
CTCTAGGGGAGAAGGTCACCATGAGCTGCAGGGCCAGCTC
AAGTGTAAATTACATATTCTGGTACCAGCAGAAGTCAGAT
GCCTCCCCCAAACTATGGATTTATTACACATCCAACCTGG
CTCCTGGAGTCCCAGCTCGCTTCAGTGGCAGTGGGTCTGG
GAACTCTTATTCTCTCACAATCAGCAGCATGGAGGGTGAA
GATGCTGCCACTTATTACTGCCAGCAGTTTACTAGTTCCC
CATTCACGTTCGGCTCGGGGACAAAGTTGGAAATAAAACG
GGGAGGAGGAGGCTCCGGCGGAGGAGGCTCTGGAGGAGGA
GGCAGCAAGGTCCAGCTGCAGCAGTCTGGAGCTGGGCTGG
TGAAACCCGGGGCATCAGTGAAGCTGTCCTGCAAGGCTTC
TGGCTACACCTTCACTGACTCTATTTTACACTGGCTAATG
CAGAGATCTGGACAGGGTCTTGAGTGGATTGGGTGGTTTT
ACCCTGGAAGTGGTAGTATAAAGTACAATGAGAAATTCAA
GGACAAGGCCACATTGACTGCGGACAAGTCCTCCAGCACA
GTCTATATGGAGCTTAGTAGATTGACATCTGAAGACTCTG
CGTTCTATTTCTGTGCAAGGCACGAAGATGGTTACGACGG
GTTTGCTTACTGGGGCCAAGGGACTCTGGTCACTGTCTCT
GCA
(SEQ ID NO: 65)
GATGTCCAGATAACCCAGTCTCCATCTTATCTTGCTGCAT
CTCCTGGAGAAACCATTACTATTAATTGCAGGGCAAGTAA
GACCATTAGCAAATATTTGGCCTGGTATCAAGAGAAACCT
GGGAAAACTAATAAGCTTCTTATCTACTCTGGATCCACTT
TGCAATCTGGAATTCCATCAAGGTTCAGTGGCAGTGGATC
TGGTACAGATTTCACTCTCACCATCAGTAGCCTGGAGCCT
GAAGATTTTGCAATGTATTACTGTCAACAACATAATGAAT
ACCCGTGGACGTTCGGTGGAGGCACCAAGCTGGAAATCAA
ACGGGGAGGAGGAGGCTCCGGCGGAGGAGGCTCTGGAGGA
GGAGGCAGCCAGGTCCAACTGCAGCAGCCTGGGGCTGAGC
TGGTGAGGCCTGGGGCTTCAGTGAAGCTGTCCTGCAAGGC
TTCTGGCTACACCTTCAGCAGCTACTGGATAAACTGGGTG
AAGCAGAGGCCTGGACAAGGCCTTGAGTGGATCGGAAATA
TTTATCCTTCTGATAGTTATACTAACTACAATCAAAAGTT
CAAGGACAAGGCCACATTGACTGTAGACAAATCCTCCAGT
ACAGCCTACATGCAGCTCAGCAGCCCGACATCTGAGGACT
CTGCGGTCTATTACTGTACAAGAGAGGGGCATTACTACGG
ATCCTTCGGTGCTATGGACTACTGGGGTCAAGGAACCTCA
GTCACCGTCTCCTCA
(SEQ ID NO: 66)
GATGTACAGCTTCAGGAGTCAGGACCTGGCCTCGTGAAAC
CTTCTCAGTCTCTGTCTCTCACCTGCTCTGTCACTGGCTA
CTCCATCACCAGTGGTTATTACTGGAACTGGATCCGGCAG
TTTCCAGGAAACAAACTGGAATGGATGGGCTACATAAGCT
ACGACGGTAGCAATAACTACAACCCATCTCTCAAAAATCG
AATCTCCATCACTCGTGACACATCTAAGAACCAGTTTTTC
CTGAAGTTGAATTCTGTGACTACTGAGGACACAGCTACAT
ATTACTGTACAAAAGGGGGCTACGGCTACTACTTTGACTA
CTGGGGCCAAGGCACCACTCTCACAGTCTCCTCAGGAGGA
GGAGGCTCCGGCGGAGGAGGCTCTGGAGGAGGAGGCAGCG
ACATCCAGATGACTCAGTCTCCAGCCTCCCTATCTGCATC
TGTGGGAGAAACTGTCACCATCACATGTCGAGCAAGTGGG
AATATTCACAATTATTTAGCATGGTATCAGCAGAAACAGG
GAAAATCTCCTCAGCTCCTGGTCTATAATGCAAAAACCTT
AGCAGATGGTGTGCCATCAAGGTTCAGTGGCAGTGGATCA
GGAACACAATATTCTCTCAAGATCAACAGCCTGCAGCCTG
AAGATTTTGGGAGTTATTACTGTCAACATTTTTGGAGTAC
TCCTCCGACGTTCGGTGGAGGCACCAAGCTGGAAATCAAA
CGG
(SEQ ID NO: 67)
CAGGTCCAGCTGCAGCAGCCTGGGGCTGAGCTTGTGAAGC
CTGGGACTTCAGTGAAGCTGTCCTGCAAGGCTTCTGGCTA
CAACTTCACCAGCTACTGGATAAACTGGGTGAAGCTGAGG
CCTGGACAAGGCCTTGAGTGGATTGGAGATATTTATCCTG
GTAGTGGTAATACTAATTACAATGAGAAGTTCAAGAGCAA
GGCCACACTGACTGTAGACACATCCTCCACCACAGCCTAC
ATGCAACTTAGTAGCCTGGCCTCTGAGGACTCTGCTCTCT
ATTACTGTGCAAGACGGGGGTATCTTGACTACTGGGGCCA
AGGCACCACTCTCACAGTCTCCTCAGGAGGAGGAGGCTCC
GGCGGAGGAGGCTCTGGAGGAGGAGGCAGCGACATCCAGA
TGACTCAGTCTCCAGCCTCCCTATCTGCATCTGTGGGAGA
AACTGTCACCATCACATGTCGAGCAAGTGGGAATATTCAC
AATTATTTAGCATGGTATCAGCAGAAACAGGGAAAATCTC
CTCAGCTCCTGGTCTATAATGCAAAAACCTTAGCAGATGG
TGTGCCATCAAGGTTCAGTGGCAGTGGATCAGGAACACAA
TATTCTCTCACGATCAACAGCCTGCAGCCTGAAGATTTTG
GGAGTTACTACTGTCAACATTTTTGGAGTACTCCTCTCAC
GTTCGGTGCTGGGACCAAGCTGGAGCTGAAACGG
(SEQ ID NO: 68)
CAGGTTCAGCTGCAGCAGTCTGGAGCTGAGCTGATGAAGC
CTGGGGCCTCAGTGAAGATATCCTGCAAGGCTACTGGCTA
CACATTCAGTAGCTACTGGATAGAGTGGGTAAAGCAGAGG
CCTGGACATGGCCTTGAGTGGATTGGAGAGATTTTACCTG
GAAGTGGTAGTACTAACTACAATGAGAAGTTCAAGGGCAA
GGCCACATTCACTGCAGATACATCCTCCAACACAGCCTAC
ATGCAACTCAGCAGCCTGACATCTGAGGACTCTGCCGTCT
ATTACTGTGCAAGATGGGGGCAGCTCGGGCTTTTTTATGC
TATGGACTACTGGGGTCAAGGAACCTCAGTCACCGTCTCC
TCAGGAGGAGGAGGCTCCGGCGGAGGAGGCTCTGGAGGAG
GAGGCAGCGACATCCAGATGACTCAGTCTCCAGCCTCCCT
ATCTGCATCTGTGGGAGAAACTGTCACCATCACATGTCGA
GCAAGTGAGAATATTTACAGTTATTTAGCATGGTATCAGC
AGAAACAGGGAAAATCTCCTCAGCTCCTGGTCTATAATGC
AAAAACCTTAGCAGAAGGTGTGCCATCAAGGTTCAGTGGC
AGTGGATCAGGCACACAGTTTTCTCTGAAGATCAACAGCC
TGCAGCCTGAAGATTTTGGGAGTTATTACTGTCAACATCA
TTATGGTAGTCCGCTCACGTTCGGTGCTGGGACCAAGCTG
GAGCTGAAACGG
(SEQ ID NO: 69)
AAGGTCCAGCTGCAGCAGTCTGGAGCTGGGCTGGTGAAAC
CCGGGGCATCAGTGAAGCTGTCCTGCAAGGCTTCTGGCTA
CACCTTCACTGACTCTATTTTACACTGGCTAATGCAGAGA
TCTGGACAGGGTCTTGAGTGGATTGGGTGGTTTTACCCTG
GAAGTGGTAGTATAAAGTACAATGAGAAATTCAAGGACAA
GGCCACATTGACTGCGGACAAGTCCTCCAGCACAGTCTAT
ATGGAGCTTAGTAGATTGACATCTGAAGACTCTGCGTTCT
ATTTCTGTGCAAGGCACGAAGATGGTTACGACGGGTTTGC
TTACTGGGGCCAAGGGACTCTGGTCACTGTCTCTGCAGGA
GGAGGAGGCTCCGGCGGAGGAGGCTCTGGAGGAGGAGGCA
GCGAAAATGTGCTCACCCAGTCTCCAGCAATCATGTCTGC
ATCTCTAGGGGAGAAGGTCACCATGAGCTGCAGGGCCAGC
TCAAGTGTAAATTACATATTCTGGTACCAGCAGAAGTCAG
ATGCCTCCCCCAAACTATGGATTTATTACACATCCAACCT
GGCTCCTGGAGTCCCAGCTCGCTTCAGTGGCAGTGGGTCT
GGGAACTCTTATTCTCTCACAATCAGCAGCATGGAGGGTG
AAGATGCTGCCACTTATTACTGCCAGCAGTTTACTAGTTC
CCCATTCACGTTCGGCTCGGGGACAAAGTTGGAAATAAAA
CGG
(SEQ ID NO: 70)
CAGGTCCAACTGCAGCAGCCTGGGGCTGAGCTGGTGAGGC
CTGGGGCTTCAGTGAAGCTGTCCTGCAAGGCTTCTGGCTA
CACCTTCAGCAGCTACTGGATAAACTGGGTGAAGCAGAGG
CCTGGACAAGGCCTTGAGTGGATCGGAAATATTTATCCTT
CTGATAGTTATACTAACTACAATCAAAAGTTCAAGGACAA
GGCCACATTGACTGTAGACAAATCCTCCAGTACAGCCTAC
ATGCAGCTCAGCAGCCCGACATCTGAGGACTCTGCGGTCT
ATTACTGTACAAGAGAGGGGCATTACTACGGATCCTTCGG
TGCTATGGACTACTGGGGTCAAGGAACCTCAGTCACCGTC
TCCTCAGGAGGAGGAGGCTCCGGCGGAGGAGGCTCTGGAG
GAGGAGGCAGCGATGTCCAGATAACCCAGTCTCCATCTTA
TCTTGCTGCATCTCCTGGAGAAACCATTACTATTAATTGC
AGGGCAAGTAAGACCATTAGCAAATATTTGGCCTGGTATC
AAGAGAAACCTGGGAAAACTAATAAGCTTCTTATCTACTC
TGGATCCACTTTGCAATCTGGAATTCCATCAAGGTTCAGT
GGCAGTGGATCTGGTACAGATTTCACTCTCACCATCAGTA
GCCTGGAGCCTGAAGATTTTGCAATGTATTACTGTCAACA
ACATAATGAATACCCGTGGACGTTCGGTGGAGGCACCAAG
CTGGAAATCAAACGG

Wherein, in this application, the nucleotide sequences as shown in the above SEQ ID NOs: 51 and 56 encode the heavy chain variable region and light chain variable region of FC2-117, respectively, the nucleotide sequences as shown in the above SEQ ID NOs: 52 and 57 encode the heavy chain variable region and light chain variable region of FC2-070, respectively, the nucleotide sequences as shown in the above SEQ ID NOs: 53 and 58 encode the heavy chain variable region and light chain variable region of FC2-153, respectively, the nucleotide sequences as shown in the above SEQ ID NOs: 54 and 59 encode the heavy chain variable region and light chain variable region of FC2-201, respectively, and the nucleotide sequences as shown in the above SEQ ID NOs: 55 and 60 encode the heavy chain variable region and light chain variable region of FC2-203, respectively. The nucleotide sequences as shown in SEQ ID NOs: 61-65 encode the single chain antibody having the amino acid sequence as shown in SEQ ID NOs: 41-45, and the nucleotide sequences as shown in SEQ ID NOs: 66-70 encode the single chain antibody having the amino acid sequence as shown in SEQ ID NOs: 46-50.

In the third aspect of the present disclosure, there is provided an expression vector. According to the embodiments of the present disclosure, the expression vector carries the nucleic acid molecules described above. After the expression vector according to the embodiments of the present disclosure is introduced into the appropriate receptor cells, the expression of the antibody or antigen binding fragment specifically recognizing CD22 described above can be effectively achieved under the mediation of the regulatory system, thus achieving acquisition of a large amount of the antibody or antigen binding fragment thereof in vitro.

According to the embodiments of the present disclosure, the above expression vector can further include at least one of the following additional technical features:

according to the embodiments of the present disclosure, the expression vector is an eukaryotic expression vector. In turn, the expression of the antibody or antigen binding fragment thereof specifically recognizing CD22 described above in eukaryotic cells can be achieved.

In the fourth aspect of the present disclosure, there is provided a recombinant cell. According to the embodiments of the present disclosure, the recombinant cell carries the nucleic acid molecule described above, or expresses the antibody or antigen binding fragment thereof described above. The recombinant cell according to the embodiments of the present disclosure can be used for the in vitro expression and acquisition in large quantities of the antibody or antigen binding fragment thereof specifically recognizing CD22 described above.

According to the embodiments of the present disclosure, the recombinant cell described above can further include at least one of the following additional technical features:

According to the embodiments of the present disclosure, the recombinant cell is obtained by introducing the expression vector described above into a host cell.

According to the embodiments of the present disclosure, the recombinant cell is an eukaryotic cell.

According to the embodiments of the present disclosure, the recombinant cell is a mammalian cell.

In the fifth aspect of the present disclosure, there is provided a chimeric antigen receptor. According to the embodiments of the present disclosure, the chimeric antigen receptor includes an extracellular region including a heavy chain variable region and a light chain variable region as well as a CD8 hinge region of a single chain antibody, wherein the single chain antibody specifically recognizes CD22; a transmembrane region including an immunocostimulator transmembrane region; and an intracellular region including an intracellular segment of immunocostimulator and CD3 chain; wherein the heavy chain variable region and light chain variable region of the single chain antibody are as described above. The inventor found that CART cells expressing the chimeric antigen receptor according to the embodiments of the present disclosure can specifically target CD22 positive tumor cells, have excellent specific killing effect on CD22 positive tumor cells, and have higher safety on normal cells.

In the sixth aspect of the present disclosure, there is provided a CART cell. According to the embodiments of the present disclosure, the CART cell expresses the chimeric antigen receptor described above. The CART cells according to the embodiments of the present disclosure can specifically target CD22 positive tumor cells, have excellent specific killing effect on CD22 positive tumor cells, and have higher safety on normal cells.

In the seventh aspect of the present disclosure, there is provided a pharmaceutical composition. According to the embodiments of the present disclosure, the pharmaceutical composition contains at least one of the antibody described above, nucleic acid molecule described above, expression vector described above, recombinant cell described above, chimeric antigen receptor described above or CART cell described above. The antibody or expressed antibody contained in the pharmaceutical composition according to the embodiments of the present disclosure can specifically targeted to CD22. The CART cells contained in the pharmaceutical composition have excellent specific killing effect on CD22 positive tumor cells and higher safety on normal cells.

In the eighth aspect of the present disclosure, there is provided a use of the antibody described above, nucleic acid molecule described above, expression vector described above, recombinant cell described above, chimeric antigen receptor described above, CART cell described above or pharmaceutical composition described above in the preparation of a medication for treating or preventing a cancer.

According to the embodiments of the present disclosure, the cancer is B lymphocyte leukemia or B cell lymphoma.

In the ninth aspect of the present disclosure, there is provided a use of the antibody described above, nucleic acid molecule described above, expression vector described above, recombinant cell described above, chimeric antigen receptor described above, CART cell described above or pharmaceutical composition described above in the preparation of a medication for killing CD22 positive tumor cells. The medication according to the embodiments of the present disclosure has a very good specific killing function against CD22 positive tumor cells.

In the tenth aspect of the present disclosure, there is provided a kit for detecting CD22. According to the embodiments of the present disclosure, the kit for detecting CD22 includes the antibody described above. The CD22 antibody described above can specifically targeted to CD22. The kit according to the embodiments of the present disclosure can realize the specific detection of CD22. For example, when the antibody binds to a fluorescent group, the location or real-time detection of CD22 can be achieved with a fluorescent detection device.

In the eleventh aspect of the present disclosure, there is provided a use of the antibody described above, the nucleic acid molecule described above, the expression vector described above or the recombinant cell described above in the preparation of the kit for detecting CD22 or diagnosing CD22-related diseases.

In the twelfth aspect of the present disclosure, there is provided a method for treating or preventing a disease. According to the embodiments of the present disclosure, the method includes administering at least one of the following to a subject having or suspected of having a disease:
the antibody described above;
the nucleic acid molecule described above;
the expression vector described above;
the recombinant cell described above;
the chimeric antigen receptor described above;
the CART cell described above; and
the pharmaceutical composition described above,
wherein, the disease is a CD22-related disease.

According to the embodiments of the present disclosure, the CD22-related disease includes B lymphocyte leukemia and B cell lymphoma.

In the thirteenth aspect of the present disclosure, there is provided a use of the antibody described above, nucleic acid molecule described above, expression vector described above, recombinant cell described above, chimeric antigen receptor described above, CART cell described above or pharmaceutical composition described above in the prevention and/or treatment of a cancer.

According to the embodiments of the present disclosure, the cancer includes B lymphocyte leukemia and B cell lymphoma.

Additional aspects and advantages of the present disclosure will be partially given in the following description, and some will become apparent from the following description, or will be understood from the practice of the present disclosure.

### BRIEF DESCRIPTION OF THE DRAWINGS

The above and/or additional aspects and advantages of the present disclosure will become apparent and easy to understand from the description of embodiments in combination with the following drawings, wherein:

FIG. 1 shows the detection result of affinity of the antibody according to the embodiments of the present disclosure by ELISA;

FIG. 2 shows the detection result of affinity of the antibody according to the embodiments of the present disclosure by Fortebio;

FIG. 3 shows the detection result of the binding of antibody to tumor cell line according to the embodiments of the present disclosure via FAC detection;

FIG. 4 shows the result of specific binding of CD22 antibody to B cells according to the embodiment of the present disclosure;

FIG. 5 shows the schematic diagram of plasmid structure according to the embodiments of the present disclosure;

FIG. 6 shows the detection result of CART cell positive rate according to the embodiments of the present disclosure;

FIG. 7 shows the detection result of apoptosis ratio of target cells according to the embodiments of the present disclosure by Beckmanccou LTER flow cytometry; and

FIG. 8 and FIG. 9 show the detection result of concentration of IL-2 and IFN-γ in supernatant of each well according to the embodiments of the present disclosure by ELISA.

### DETAILED DESCRIPTION OF THE EMBODIMENTS

The embodiments of the present disclosure will be described in detail below. The examples of the embodiments are shown in the drawings, in which the same or similar reference numbers throughout the present disclosure represent the same or similar elements or elements with the same or similar functions. The embodiments described below with reference to the accompanying drawings are exemplary and are intended to explain the present disclosure, but cannot be understood as restrictions thereto.

In addition, the terms “first” and “second” are only used for descriptive purposes and cannot be understood as indicating or implying relative importance or implicitly indicating the number of indicated technical features. Therefore, the features defined as “first” and “second” can explicitly or implicitly include at least one such features. In the description of the present disclosure, “multiple” means at least two, such as two, three, etc., unless otherwise specifically defined.

Antibody

In this context, the term “antibody” refers to an immunoglobulin molecule that can bind to a specific antigen. It consists of two light chains with lighter molecular weight and two heavy chains with heavier molecular weight. The heavy chain (H chain) and light chain (L chain) are connected by disulfide bonds to form a tetrapeptide chain molecule. Wherein, the amino acid sequence of the amino terminal (N-end) of the peptide chain varies greatly, which is called as the variable region (V region), while the carboxyl terminal (C-end) is relatively stable and varies very little, which is called as the constant region (C region). The V regions of L chain and H chain are called as VL and VH, respectively.

The composition and arrangement order of amino acids in some regions of the variable region have a higher degree of variability, which is called as hypervariable region (HVR). The hypervariable region is the location where antigen and antibody bind, so it is also called as the complementarity-determining region (CDR). There are three CDR regions on the heavy chain variable region and the light chain variable region.

The present disclosure uses CD22 extracellular segment to obtain high specificity and high affinity Fab (antigen-binding fragment) antibody fragments against CD22 through immunization. The antibody fragment can specifically bind to CD22 antigen, which can be used to targeted treating diseases such as tumors, etc.

In some embodiments, the present disclosure provides an antibody or antigen binding fragment that can specifically recognize CD22. The antibody contains a CDR sequence selected from at least one of the following or an amino acid sequence having at least 95% identity thereto: heavy chain variable region CDR sequence as shown in SEQ ID NOs: 1-15, and a light-chain variable region CDR sequence as shown in SEQ IN NOs: 16-30. In another embodiments, the antibody or antigen binding fragment provided by the present disclosure has conservative amino acid substitution compared with the heavy chain and light chain described above. “Antigen binding fragment” refers to an antibody fragment that maintains the ability of specific binding to an antigen. “Conservative amino acid substitution” refers to the substitution of an amino acid by a biologically, chemically or structurally similar residue of another amino acid. By “biologically similarity”, it means that the substitution does not damage the CD22 antibody or the biological activity of a CD22 antigen. By “structurally similarity”, it means that amino acids have side chains with similar length, such as alanine, glycine or serine, or have side chains with similar size. By “chemically similarity”, it means that amino acids have the same charges or are both hydrophilic or hydrophobic. For example, hydrophobic residues such as isoleucine, valine, leucine or methionine are mutually substituted. Alternatively, polar amino acids are mutually substituted, such as substituting arginine for lysine, substituting glutamate for aspartate, substituting glutamine for asparagine, and substituting serine for threonine, etc.

In some embodiments, the present disclosure provides an antibody or antigen binding fragment, the antibody or antigen binding fragment has a heavy chain variable region of the amino acid sequence as shown in any one of SEQ ID NOs: 31-35 and a light chain variable region of the amino acid sequence as shown in any one of SEQ ID NOs: 36-40. The inventor can obtain the CDR region of the above heavy chain variable region sequence (as shown in SEQ ID NOs: 1-15) and the CDR region of the light chain variable region sequence (as shown in SEQ ID NOs: 16-30) through the antibody sequence alignment database (NCBI, and IMGT). In another embodiments, the heavy chain variable region sequence of the antibody or antigen binding fragment has conservative amino acid substitutions compared with the amino acid sequence as shown in SEQ ID NOs: 31-35. In another embodiments, the light chain variable region sequence of the antibody or antigen binding fragment has conservative amino acid substitutions compared with the amino acid sequence as shown in any one of SEQ ID NOs: 36-40. Of course, these conservative amino acid substitutions will not change the biological functions of the antibody or antigen binding fragment. In some specific embodiments, these conservative amino acid substitutions can occur on amino acids other than the CDR regions in the heavy chain variable region and the light chain variable region.

In some preferred embodiments, the present disclosure provides an anti CD22 antibody. The light chain constant region and heavy chain constant region of the antibody are both derived from human-derived IgG1, 2 or 4. Furthermore, the immunogenicity of the antibody can be effectively reduced. In some preferred embodiments, the present disclosure provides an anti CD22 single chain antibody, which has the amino acid sequence as shown in SEQ ID NOs: 41-50.

Nucleic Acid Molecule, Expression Vector, and Recombinant Cell

In the process of preparing or obtaining these antibodies, nucleic acid molecules expressing these antibodies can be connected with different vectors, and then expressed in different cells to obtain corresponding antibodies.

To this end, the present disclosure also provides an isolated nucleic acid molecule encoding the antibody or antigen binding fragment described above.

In some embodiments, the isolated nucleic acid molecule has a nucleotide sequence as shown in any one of SEQ ID NOs: 51-55 or has a nucleotide sequence as shown in any one of SEQ ID NOs: 56-60 or has a nucleotide sequence as shown in SEQ ID NOs: 61-70.

In some embodiments, the isolated nucleic acid molecule has at least 90% homology or higher, preferably 95% homology or higher, and more preferably 98% and 99% homology or higher, to the nucleotide sequence as shown in SEQ ID NOs: 51-55 described above. In at least some embodiments, the isolated polynucleotide has at least 90% homology or higher, preferably 95% homology or higher, and more preferably 98% and 99% homology or higher, to the nucleotide sequence as shown in SEQ ID NOs: 56-60. In at least some embodiments, the isolated polynucleotide has at least 90% homology or higher, preferably 95% homology or higher, and more preferably 98% and 99% homology or higher, to the nucleotide sequence as shown in SEQ ID NOs: 61-70. These sequences having homology to the nucleotide sequences as shown in SEQ ID NOs: 51-55 or SEQ ID NOs: 56-60 or SEQ ID NOs: 61-70 can express amino acids similar to SEQ ID NOs: 31-35 and SEQ ID NOs: 36-40 or amino acid sequences similar to SEQ ID NOs: 41-50, so that they can specifically bind to CD22 antigen to achieve the targeted function of antibody.

In some embodiments, the isolated nucleic acid molecule has nucleotide sequences of heavy chain variable region as shown in SEQ ID NOs: 51-55 and nucleotide sequences of light chain variable region as shown in SEQ ID Nos: 56-60. These nucleotide sequences are more easily expressed in mammalian cells after species optimization.

The present disclosure also provides an expression vector containing the isolated nucleic acid molecules described above. When the isolated polynucleotides described above are connected to a vector, the polynucleotides can be directly or indirectly connected to the control elements on the vector, as long as these control elements can control the translation and expression of the polynucleotides. Of course, these control elements can directly come from the vector itself, or they can be exogenous, that is, they do not come from the vector itself. Of course, the polynucleotide can be operatively connected with the control element. By “operatively connected” as used herein, it refers to the connection of exogenous genes to the vector, so that the control elements in the vector, such as transcription control sequence and translation control sequence, etc., can play their expected functions of regulating the transcription and translation of exogenous genes. Of course, the polynucleotides used to encode the heavy chain and light chain of antibody can be each independently inserted into different vectors, usually into the same vector. Common vectors can be for example, plasmids and phages, etc. For example, Plasmid-X plasmids.

The present disclosure also provides a recombinant cell containing the expression vector therein. The expression vector can be introduced into mammalian cells to construct the recombinant cells, and then these recombinant cells can be used to express antibodies or antigen binding fragments provided in the present disclosure. By culturing with the recombinant cells, the corresponding antibodies can be obtained. These available mammalian cells can be for example, CHO cells, etc.

Chimeric Antigen Receptor and CAR T Cells

The present disclosure relates to a chimeric antigen receptor (CAR), which is a molecule that combines the specificity of an antibody-based targeting desired antigen (for example, a tumor antigen) with a T-cell receptor activated intracellular domain to generate a chimeric protein that exhibits specific anti-tumor cell immune activity.

CAR expressing T cells are called as CAR T cells or CAR modified T cells.

In one embodiment, the CARs of the present disclosure include an extracellular region, a transmembrane region and an intracellular region with an antigen recognition domain.

The CAR of the embodiments of the present disclosure (including functional parts and functional variants thereof) can be obtained by methods known in the art. CARs may be prepared by any suitable methods for the preparation of peptides or proteins. Suitable methods for de novo synthesis of peptides and proteins are described in the references, e.g. Chan et al., Fmoc Solid Phase Peptide Synthesis, Oxford University Press, Oxford, United Kingdom, 2000; Peptide and Protein Drug Analysis, Reid, R. edited, Marcel Dekker Inc., 2000; Epitope Mapping, Westwood et al., edited, Oxford University Press, Oxford, United Kingdom, 2001; and U.S. Pat. No. 5,449,752. In addition, peptides and proteins can be produced via recombination by standard recombination methods using the nucleic acids as described herein. See, for example, Sambrook, et al., Molecular Cloning: A Laboratory Manual, 3rd edition, Cold Spring Harbor Press, Cold Spring Harbor, N Y 2001; and Ausubel et al., Current Protocols in Molecular Biology, Greene Publishing Associates and John Wiley & Sons, N Y, 1994. In addition, some CARs of the present disclosure (including functional parts and functional variants thereof) can be isolated and/or purified from sources such as plants, bacteria, insects, and mammals such as rats and humans, etc. The isolation and purification methods are well known in the art. Optionally, the CARs as described herein (including functional parts and functional variants thereof) can be synthesized commercially by Companies Synpep (Dublin, CA), Peptide Technologies Corp. (Gaithersburg, MD) and Multiple Peptide Systems (San Diego, CA). In this regard, the CARs of the present disclosure can be synthesized, recombined, isolated and/or purified.

The method for testing the ability of antigen to bind to any functional parts of the CARs of the present disclosure is known in the art and includes any antibody-antigen binding assays, such as radioimmunoassay (RIA), ELISA, Western blotting, immunoprecipitation and competitive inhibition assay (see, for example, Janeway et al., the same as below, and U.S. Patent Application No. 2002/0197266 A1).

The functional variants of CARs of the present disclosure as described herein are also included within the scope of the present disclosure. The term “functional variants” as used herein refers to CARs, polypeptides or proteins that have a large amount of or significant sequence identity or similarity to the parent CARs, and the functional variants retain the biological activity of the CAR variants. Functional variants include, for example, those variants of CARs (parent CARs) as described herein, which retain the ability to recognize target cells to the extent that is similar to, the same as or higher than that of the parent CARs. With respect to the parent CAR, the amino acid sequence of the functional variant can have, for example at least about 30%, about 50%, about 75%, about 80%, about 90%, about 98%, about 99% identity or higher to the parent CARs.

The functional variant may, for example, comprise an amino acid sequence of a parent CAR with at least one conservative amino acid substitution. Alternatively or additionally, the functional variant may comprise an amino acid sequence of parent CARs with at least one nonconservative amino acid substitution. In this case, the nonconservative amino acid substitution without interfering with or inhibiting the biological activity of the functional variant is preferred. Nonconservative amino acid substitution can enhance the biological activity of the functional variant, making the biological activity of the functional variant increase compared with the parent CARs.

The amino acid substitution of CARs in the present disclosure is preferably a conservative amino acid substitution. Conservative amino acid substitution is known in the art and includes an amino acid substitution in which an amino acid with certain physical and/or chemical properties is exchanged for another amino acid with the same or similar chemical or physical properties. For example, the conservative amino acid substitution can be to replace an acidic/negatively charged polar amino acid with another acidic/negatively charged polar amino acid (such as Asp or Glu), replace an amino acid having a non-polar side chain with another amino acid having a non-polar side chain (such as Ala, Gly, Val, He, Leu, Met, Phe, Pro, Tip, Cys, Val, etc.), replace a basic/positively charged polar amino acid with another basic/positively charged polar amino acid (such as Lys, His, Arg, etc.), replace a non-charged amino acid having a polar side chain with another non-charged amino acid having a polar side chain (such as Asn, Gln, Ser, Thr, Tyr, etc.), replace an amino acid having a R branched side chain with another amino acid having a R branched side chain (such as Ile, Thr and Val), and replace an amino acid having an aromatic side chain with another amino acid having an aromatic side chain (such as His, Phe, Trp and Tyr), etc.

The CARs of the embodiments of the present disclosure (including functional parts and functional variants of the present disclosure) can contain synthetic amino acids in place of one or more naturally occurring amino acids. Such synthetic amino acids are known in the art and include, for example, aminocyclohexane carboxylic acid, n-leucine, α-amino n-decanoic acid, homoserine, S-acetamidomethyl cysteine, trans-3- and trans-4-hydroxyproline, 4-aminophenylalanine, 4-nitrophenylalanine, 4-chlorophenylalanine, 4-carboxyphenylalanine, 3-phenylserine, β-hydroxyphenylalanine, phenylglycine, α-naphthylalanine, cyclohexylalanine, cyclohexylglycine, indolin-2-carboxylic acid, 1,2,3,4-tetrahydroisoquinoline-3-carboxylic acid, aminomalonic acid, aminomalonic acid monoamide, N′-benzyl-N′-methyl lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norcamphene)-carboxylic acid, α,γ-diaminobutyric acid, α,β-diaminopropionic acid, homophenylalanine, and α-tertbutyl glycine.

Pharmaceutical Composition, Kit, Pharmaceutical Use and Use in Preparation of Kit

The present disclosure also provides a pharmaceutical composition, which includes the antibody or antigen binding fragment described above and a pharmaceutically acceptable carrier.

The anti-CD22 antibody provided herein can be incorporated into a pharmaceutical composition which is suitable for administration to a subject. Generally, these pharmaceutical compositions include anti-CD22 antibodies provided herein and a pharmaceutically acceptable carrier. The “pharmaceutically acceptable carrier” can include any and all solvents, dispersion media, coating, antibacterial and antifungal agents, isotonic agents and delayed absorbents that are physiologically compatible. Specific examples can be one or more of water, brine, phosphate buffer brine, glucose, glycerin, ethanol, etc. and their compositions. In many cases, the pharmaceutical composition includes isotonic agents, such as sugars, polyols (such as mannitol and sorbitol) or sodium chloride, etc. Of course, pharmaceutically acceptable carriers can also include trace auxiliary substances, such as wetting agents or emulsifiers, preservatives or buffers, so as to extend the shelf life or efficacy of antibodies.

For example, the antibody of the present disclosure can be incorporated into a pharmaceutical composition which is suitable for parenteral administration (e.g., intravenous, subcutaneous, intraperitoneal, and intramuscular). These pharmaceutical compositions can be prepared into various forms. For example, liquid, semi-solid and solid dosage forms, etc., include but are not limited to liquid solutions (such as injection solutions and infusion solutions), dispersions or suspensions, tablets, pills, powders, liposomes and suppositories. Typical pharmaceutical compositions are in the form of injection solutions or infusion solutions. The antibody can be administered by intravenous infusion or injection or intramuscular or subcutaneous injection.

Of course, the anti-CD22 antibody herein can also be made into a kit or part of other diagnostic reagents as needed. According to the embodiments of the present disclosure, there is also provides a kit, which comprises the CD22 antibody described above. The kit provided by the present disclosure can be, for example, used for immunoblotting, immunoprecipitation, etc., which involves the use of CD22 antigen and antibody specific binding properties to detect etc. These kits can include any one or more of the following: an antagonist, an anti-CD22 antibody or a drug reference material; protein purification column; immunoglobulin affinity purification buffer; diluent for cell assay; an instruction or a document, etc. Anti-CD22 antibodies can be used for different types of diagnostic tests, for example for the detection of presence of various diseases or drugs, toxins or other proteins, etc. in vitro or in vivo. For example, it can be used to test related diseases by testing the serum or blood of the subject. Such related diseases can include CD22 related diseases, such as a cancer, etc. Of course, the antibodies provided herein can also be used for radioimmunoassay and radioimmunotherapy of the diseases described above.

These cancers or tumors can be any kind of uncontrolled cell growth. Specifically, it can be B lymphocyte leukemia or B cell lymphoma.

When using the anti-CD22 antibody or CART provided by the present disclosure to treat the diseases described above, it is only needed to provide the anti-CD22 antibody or CART cells provided by the present disclosure to the subject. To this end, the present disclosure provides a method for treating the diseases described above, including administering the antibody or antigen binding fragment thereof CART cells provided by the present disclosure to a subject in need thereof.

The present disclosure has the following advantages:

1) the present disclosure obtained a fully new CD22 antibody by immunizing mice, the antibody has high affinity and strong specificity, and CART cells constructed based on this sequence have a very good specific killing function against CD22 positive tumor cells in vitro.

2) CART developed based on the antibody sequence obtained according to the present disclosure can specifically kill the CD22 positive tumor cells, and can be applied to immunotherapy of patients with B lymphocyte leukemia and B cell lymphoma; the existing clinical results show that the efficacy of immunocyte therapy is superior to the current treatment means for patients with B lymphocyte leukemia and B-cell lymphoma, which plays a great role in promoting the treatment of patients with B lymphocyte leukemia and B-cell lymphoma.

The embodiments of the present disclosure will be explained below in combination with examples. Those skilled in the art will understand that the following examples are only used to explain the present disclosure, and should not be considered as limiting the scope of the present disclosure. If no specific technology or conditions are indicated in the examples, the technology or conditions described in the literature in the art or the product specification shall be followed. The reagents or instruments used of which the manufacturers are not indicated are conventional products that are commercially available.

### Example 1 Acquisition of Targeted CD22 Antibody

Human CD22 extracellular segment conjugated His label (hereinafter referred to as h CD22-His, ACRO, CD2-H52H8) was intraperitoneally injected into BALB/c mice (Guangdong Medical Laboratory Animal Center) at 100 μg/200 μl/week/mouse/time. After 3 weeks of immunization, blood of mice was taken at tail every week and the expression of CD22 antibody in serum was detected; mice with high expression level of CD22 antibody in serum were selected, and spleen cells and tumor cells (SP20, ATCC HB-12546) were taken, fused to form fusants, after 10-14 days of culture, the fusants expressing CD22 antibody in the culture supernatant were selected for monoclonal cloning; monoclonal hybridoma cell strains expressing CD22 antibody were selected for expanded culture, after 7-10 days of culture, the cell culture medium was collected for purification to obtain the CD22 antibody, 5 candidate CD22 antibodies obtained were sequenced, and the sequencing results were shown below:

Amino acid sequence of light chain variable region (CDR sequence was shown in bold and underlined):

FC2-070:
DIQMTQSPASLSASVGETVTITCRASGNIHNYLAWYQQKQGKSPQLLVYNAKTLADGVPSRF
SGSGSGTQYSLTINSLQPEDFGSYYCQHFWSTPLTFGAGTKLELKR
FC2-117:
DIQMTQSPASLSASVGETVTITCRASGNIHNYLAWYQQKQGKSPQLLVYNAKTLADGVPSRF
SGSGSGTQYSLKINSLQPEDFGSYYCQHFWSTPPTFGGGTKLEIKR
FC2-153:
DIQMTQSPASLSASVGETVTITCRASENIYSYLAWYQQKQGKSPQLLVYNAKTLAEGVPSRFS
GSGSGTQFSLKINSLQPEDFGSYYCQHHYGSPLTFGAGTKLELKR
FC2-201:
ENVLTQSPAIMSASLGEKVTMSCRASSSVNYIFWYQQKSDASPKLWIYYTSNLAPGVPARFSG
SGSGNSYSLTISSMEGEDAATYYCQQFTSSPFTFGSGTKLEIKR
FC2-203:
DVQITQSPSYLAASPGETITINCRASKTISKYLAWYQEKPGKTNKLLIYSGSTLQSGIPSRFS
GSGSGTDFTLTISSLEPEDFAMYYCQQHNEYPWTFGGGTKLEIKR
Amino acid sequence of heavy chain variable region
(CDR sequence was shown in bold and underlined):
FC2-070:
QVQLQQPGAELVKPGTSVKLSCKASGYNFTSYWINWWKLRPGQGLEWIGDIYPGSGNTNYN
EKFKSKATLTVDTSSTTAYMQLSSLASEDSALYYCARRGYLDYWGQGTTLTVSS;
FC2-117:
DVQLQESGPGLVKPSQSLSLTCSVTGYSITSGYYWNWIRQFPGNKLEWMGYISYDGSNNYN
PSLKNRISITRDTSKNQFFLKLNSVTTEDTATYYCTKGGYGYYFDYWGQGTTLTVSS;
FC2-153:
QVQLQQSGAELMKPGASVKISCKATGYTFSSYWIEWWKQRPGHGLEWIGEILPGSGSTNYN
EKFKGKATFTADTSSNTAYMQLSSLTSEDSAVYYCARWGQLGLFYAMDYWGQGTSVTVSS;
FC2-201:
KVQLQQSGAGLVKPGASVKLSCKASGYTFTDSILHWLMQRSGQGLEWIGWFYPGSGSIKYN
EKFKDKATLTADKSSSTVYMELSRLTSEDSAFYFCARHEDGYDGFAYWGQGTLVTVSA;
FC2-203:
QVQLQQPGAELVRPGASVKLSCKASGYTFSSYWINWWKQRPGQGLEWIGNIYPSDSYTNYN
QKFKDKATLTVDKSSSTAYMQLSSPTSEDSAVYYCTREGHYYGSFGAMDYWGQGTSVTVSS

### Example 2 Screening of CD22 Antibodies

1) Detection of Antibody Subtypes:

The affinity of different antibodies against CD22 was determined by ELISA, the specific procedure was as follows:

Coating the hCD22-His in 96-well enzyme-linked coating plate, at a concentration of 2 μg/ml, 100 μl/well, combining 10 μg/ml of antibody with a recombinant protein, using secondary antibody anti Mouse IgG1-HR, anti Mouse IgG2a-HRP, anti Mouse IgG2b-HRP, anti Mouse IgG3-HRP, anti Mouse IgG-HRP, and anti Mouse IgM-HRP and developing to detect the OD450 values of each antibody (the specific operation steps were general ELISA operation steps) S-HCL-1 was a commercial CD22 antibody of mlgG2b subtype; RFB-4 is a commercial CD22 antibody of mIgG1 subtype; M971 is a humanized CD22 antibody of mIgG1 subtype. The specific sequence and information can be found publicly. The antibody was synthesized by Shenzhen FeipengBiological therapy Co., LTD. The results show that the CD22 antibody Fc2-070 was mlgG2b subtype, Fc2-117 was mIgG1 subtype, Fc2-153 was mIgG1 subtype, Fc2-201 was mlgG2b subtype, and Fc2-203 was mIgG1 subtype.

2) Affinity Test:

The affinity of different antibodies against CD22 was determined by three methods of ELISA, Fortebio and FACs, the specific procedures were as follows:

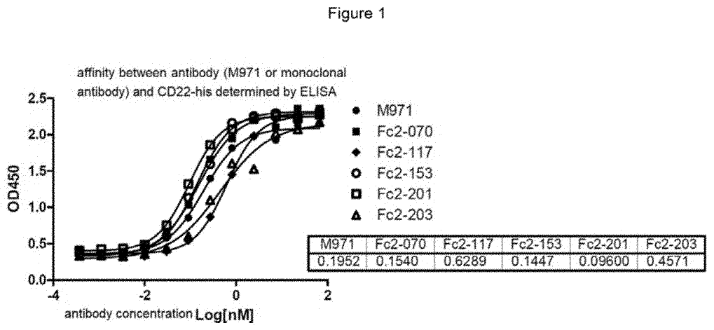

Detection of antibody affinity by ELISA: coating h CD22-His in 96-well enzyme-linked coating plate at a concentration of 2 μg/ml, 100 dal/well, and combining the antibody at a 3-fold gradient dilution with antigen to detect EC50 of each antibody (the specific operation steps were the general ELISA operation steps). M971 was a 7 epitope humanized CD22 antibody. The specific sequence and information can be found publicly. The antibody was synthesized by Shenzhen FeipengBiological therapy Co., LTD. The results are shown in FIG. 1 below. The results show that the EC50 of CD22 antibodies M971, Fc2-070, Fc2-117, Fc2-153, Fc2-201 and Fc2-203 are at the same level.

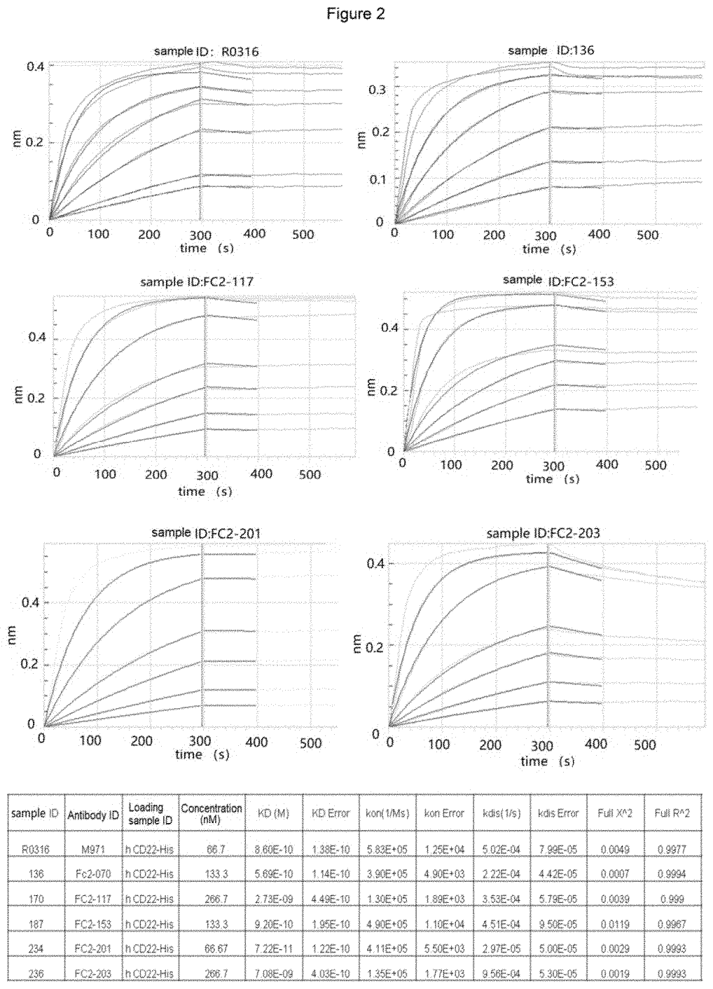

Detection of antibody affinity by Fortebio: using ProA biosensor, first loading buffer, then adding 5 μg/mL of h CD22-His, and then loading 6 antibodies, respectively, and detecting KD, Kon and Kd is of 6 antibodies, respectively. The specific operation steps can be understood by the experiment personnels who have used Fortebio instrument. The test results are shown in FIG. 2 below.

The detection of the combination of antibodies and tumor cell lines by FACs:

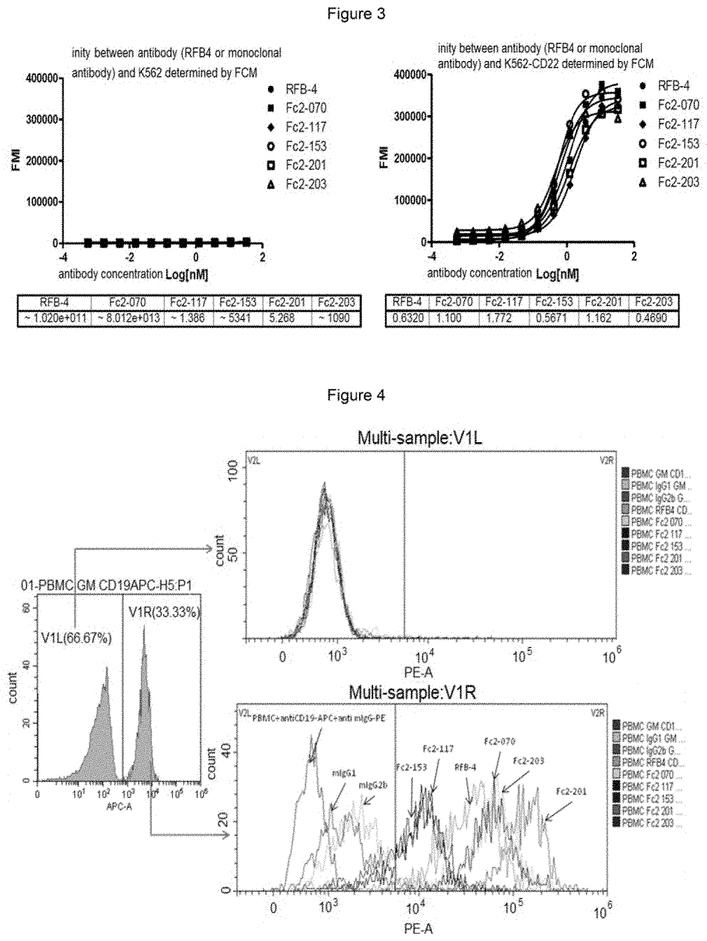

K562 cells were human chronic myeloid leukemia cells, and K562-CD22 cells were used to construct overexpressed CD22 cell lines. The specific detection methods were as follows: harvesting cells, washing with PBS once, resuspending with PBS at 1E+6 cells/ml/200 μl, incubating the antibody after gradient dilution with cells at 4° C. for 30 min, wherein the initial concentration of the antibody was 10 μg/ml, at 3-fold dilution, a total of 9 gradients, then incubating same with PE labeled anti-mouse IgG secondary antibody, washing twice, and detecting by Beckmanccou LTER flow cytometry. As shown in FIG. 3 below, RFB-4, Fc2-070, Fc2-117, Fc2-153, Fc2-201, and Fc2-203 bound to K562 and K562-CD22 cells in a concentration gradient dependent manner.

3) CD22 Antibody Specificity

Detection of specificity of CD22 antibody by flow cytometry: taking PBMC 5.0*10\hat{ }5 cells/group from volunteers, adding mouse monoclonal antibody, isotype control, and positive control antibody thereto, respectively at a final concentration of 10 μg/ml, incubating at 4° C. for 30 min, washing with PBS twice, adding secondary antibody Goat anti Mouse IgG-PE and incubating at 4° C. for 30 min, washing with PBS twice, adding anti-hCD19-APC antibody, incubating at 4° C. for 30 min, and washing with PBS once, detecting with Beckmanccou LTER flow cytometry. The detection results are shown in FIG. 4 below. Specific binding of Fc2-070, Fc2-117, Fc2-153, Fc2-201, and Fc2-203 to B cells was detected by flow cytometry.

### Example 3: Construction of CD22 CART Cells and Verification of Functions In Vitro

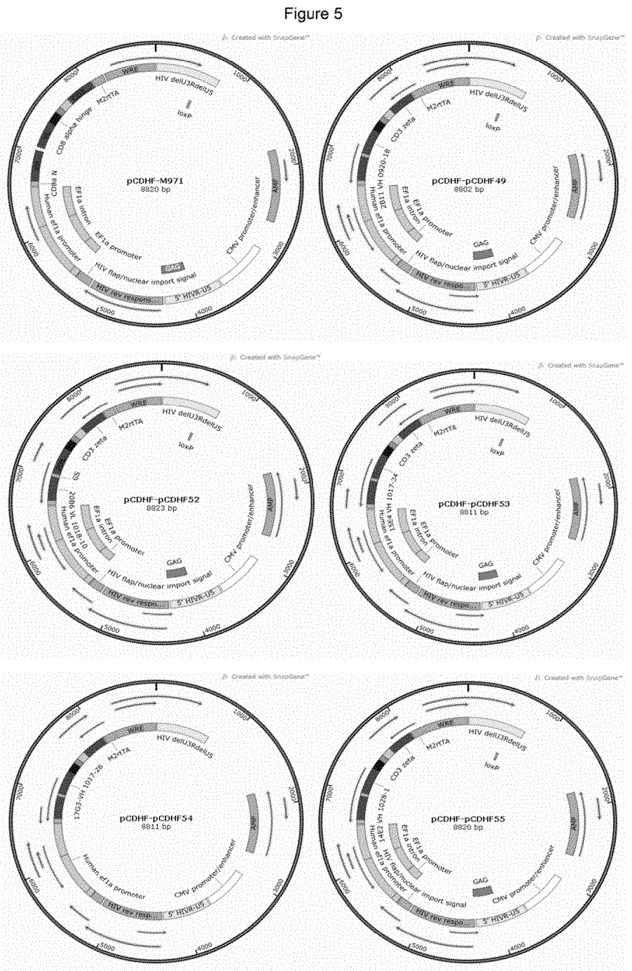

Hybridoma cell strains expressing Fc2-070, Fc2-117, Fc2-153, Fc2-201, and Fc2-203 were resuscitated. After 72 hours of normal culture, the cells were lysed to extract RNA (extraction kit: TOYOBO LIFE SCIENCE, Article No. 836700) according to the extraction steps in the instruction. cDNAs were obtained via reverse transcription (Reverse transcription kit: TOYOBO LIFE SCIENCE, Article No. 11141 ES10), antibody sequences were obtained via PCR amplification (specific primers for Mouse IgG1 and IgG2b sequences) and sequencing verification was carried out. The sequencing results are as shown in part of the sequences in Example 1. After sequencing, the scFv sequences of Fc2-070, Fc2-117, Fc2-153, Fc2-201, and Fc2-203 antibodies were constructed inlentiviral vectors to obtain CAR plasmids. The corresponding CarT plasmids were numbered as Fc2-070 (pCDHF49), Fc2-117 (pCDHF54), Fc2-153 (pCDHF55), Fc2-201 (pCDHF53) and Fc2-203 (pCDHF52). The structural representation of plasmids is as shown in FIG. 5. 293T cells were used to package lentivirus, and the acquired lentivirus will infect T cells at MOI=5:1 to prepare CART cells (the method for detecting lentivirus titer via secondary antibody APC Goat anti Mouse IgG(H+L) or fluorescent antigen hCD22-FITC Flow Cytometer and the method for preparing CART cells by infecting T cells with lentivirus can be obtained via public ways), then the in vitro function verification was carried out, and the results show that the in vitro function of CART cells constructed from the scFv sequence of CD22 antibody obtained in the present disclosure was consistent with that of CART cells constructed from the scFv sequence of positive control antibody M971.

2) Detection of CART Cell Positive Rate

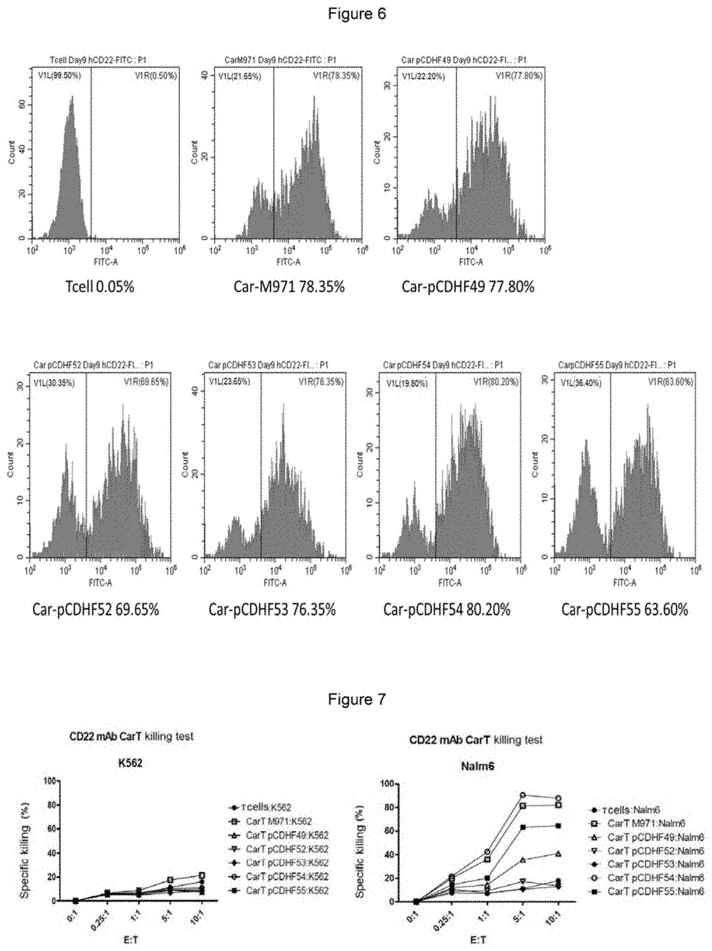

5*10E+05 cells of T cells or CarT cells were taken. After removing magnetic beads, 2 μl of fluorescent antigen hCD22-FITC was added to 100 μl of PBS system and incubated at room temperature while keeping in dark place for 15 min. After the completion of incubation, it was washed with PBS once, 200 μl of PBS was added to rescreen cells and then the cell positive rate was detected by flow cytometry. The detection results are as shown in FIG. 6.

3) Efficacy Evaluation of CART Cells In Vitro

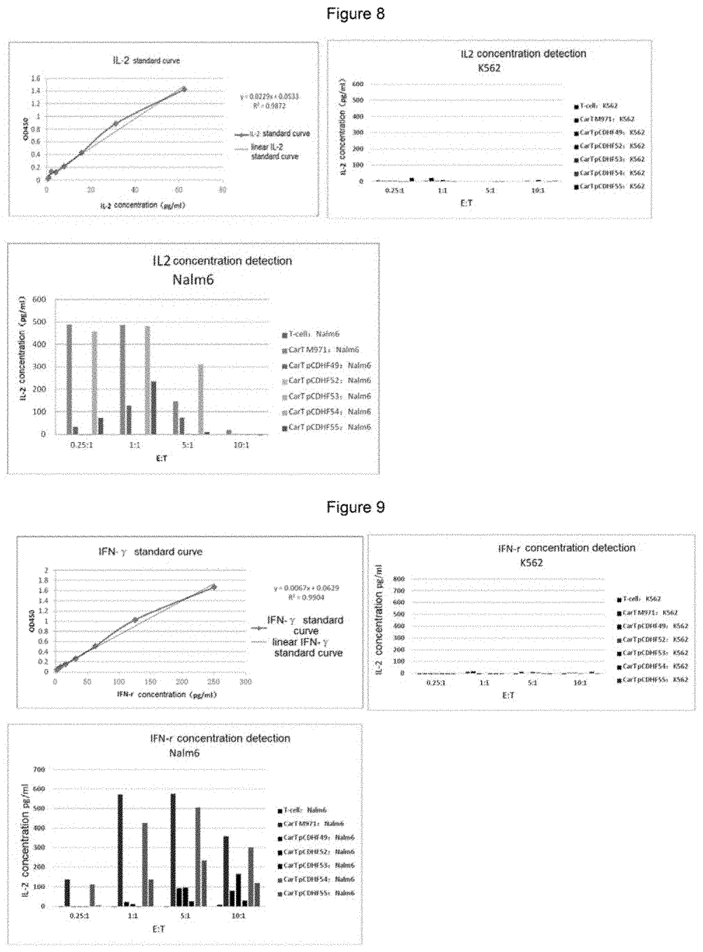

3*10E+06 cells of two target cells such as K562 and Nalm6, were taken separately, the target cells were first stained with Cytocalcein™ violet 550 at 1*10E+05 cells/100 μl/well, and the effector cells (CAR+CART, with T cells as the control) and the target cells were added into a 96-well plate at 0.25:1, 1:1, 5:1 and 10:1 and mixed evenly, with the final volume of 200 μl, after 8 hours of co-culture, the cells were mixed evenly and centrifuged. The supernatant was detected for IL-2 and IFN-γ using Human IL-2 and Human IFN gamma ELISA ELISA kit, the precipitation portion was resuspended with 100 μl of binding buffer, centrifuged at 300 g for 5 min, 2.0 μl of APC Annexin V and 1.2 μl of PI dyes were added thereto, incubating in dark place for 15 min, resuspended with 100 μl of binding buffer, and the apoptosis ratio of each target cell was detected by Beckmanccou LTER flow cytometry (as shown in FIG. 7), and the supernatant in each well was detected for the concentration of IL-2 and IFN-γ by ELISA (as shown in FIG. 8 and FIG. 9). Wherein, K562 was the CD22 negative cell and Nalm6 was the CD22 positive cell. The results show that CAR-pCDHF49, CAR-pCDHF54, CAR-pCDHF55, CAR-pCDHF53, CAR-pCDHF52 were less effective and safer than CarT-M971 in killing normal cells. Moreover, under the same cytokine secretion capacity as CarT-M971, CAR-pCDHF54 has stronger specific killing effect on CD22 positive target cells than the positive control CarT-M971.

In the description of this specification, reference to the description of the terms “one embodiment”, “some embodiments”, “examples”, “specific examples”, or “some examples” means that the specific features, structures, materials, or features described in combination with this embodiment or example are included in at least one embodiment or example of the present disclosure. In this specification, the schematic expressions of the above terms do not have to refer to the same embodiments or examples. Moreover, the specific features, structures, materials or features described can be combined in an appropriate manner in any one or more embodiments or examples. In addition, those skilled in the art can integrate and combine the different embodiments or examples described in this specification and the characteristics of different embodiments or examples without contradiction.

Although the examples of the present disclosure have been shown and described above, it can be understood that the above examples are exemplary and cannot be constructed as a limitation to the present disclosure. Those skilled in the art can make changings, modifications, substitutions and variations to the above examples within the scope of the present disclosure.

What is claimed is:

1. An antibody or antigen binding fragment thereof that can specifically recognize CD22, wherein the antibody comprises a CDR sequence selected from at least one of the following or an amino acid sequence having at least 95% identity thereto:
heavy chain variable region CDR sequence as shown in SEQ ID NOs: 1-15,
light chain variable region CDR sequence as shown in SEQ ID NOs: 16-30.

2. The antibody or antigen binding fragment thereof according to claim 1, wherein the antibody comprises:
heavy-chain variable region sequences CDR1, CDR2 and CDR3 as shown in SEQ ID NOs: 1, 2 and 3, or amino acid sequence having at least 95% identity to SEQ ID NO: 1, 2 and 3, respectively; or
heavy-chain variable region sequences CDR1, CDR2 and CDR3 as shown in SEQ ID NOs: 4, 5 and 6, or amino acid sequence having at least 95% identity to SEQ ID NO: 4, 5 and 6, respectively; or
heavy-chain variable region sequences CDR1, CDR2 and CDR3 as shown in SEQ ID NOs: 7, 8 and 9, or amino acid sequence having at least 95% identity to SEQ ID NO: 7, 8 and 9, respectively; or
heavy-chain variable region sequences CDR1, CDR2 and CDR3 as shown in SEQ ID Nos: 10, 11 and 12, or amino acid sequence having at least 95% identity to SEQ ID NO: 10, 11 and 12, respectively; or
heavy-chain variable region sequences CDR1, CDR2 and CDR3 as shown in SEQ ID Nos: 13, 14 and 15, or amino acid sequence having at least 95% identity to SEQ ID NO: 13, 14 and 15, respectively.

3. The antibody or antigen binding fragment thereof according to claim 1, wherein the antibody comprises:
light-chain variable region sequences CDR1, CDR2 and CDR3 as shown in SEQ ID NOs: 16, 17 and 18, or amino acid sequence having at least 95% identity to SEQ ID NO: 16, 17 and 18, respectively; or
light-chain variable region sequences CDR1, CDR2 and CDR3 as shown in SEQ ID Nos: 19, 20 and 21, or amino acid sequence having at least 95% identity to SEQ ID NO: 19, 20 and 21, respectively; or
light-chain variable region sequences CDR1, CDR2 and CDR3 as shown in SEQ ID Nos: 22, 23 and 24, or amino acid sequence having at least 95% identity to SEQ ID NO: 22, 23 and 24, respectively; or
light-chain variable region sequences CDR1, CDR2 and CDR3 as shown in SEQ ID Nos: 25, 26 and 27, or amino acid sequence having at least 95% identity to SEQ ID NO: 25, 26 and 27, respectively; or
light-chain variable region sequences CDR1, CDR2 and CDR3 as shown in SEQ ID Nos: 28, 29 and 30, or amino acid sequence having at least 95% identity to SEQ ID NO: 28, 29 and 30, respectively.

4. The antibody or antigen binding fragment thereof according to claim 1, wherein the antibody comprises:
heavy-chain variable region sequences CDR1, CDR2 and CDR3 as shown in SEQ ID NOs: 1, 2 and 3, or amino acid sequence having at least 95% identity to SEQ ID NO: 1, 2 and 3, respectively, and light-chain variable region sequences CDR1, CDR2 and CDR3 as shown in SEQ ID Nos: 16, 17 and 18, or amino acid sequence having at least 95% identity to SEQ ID NO: 16, 17 and 18, respectively; or
heavy-chain variable region sequences CDR1, CDR2 and CDR3 as shown in SEQ ID Nos: 4, 5 and 6, or amino acid sequence having at least 95% identity to SEQ ID NO: 4, 5 and 6, respectively, and light-chain variable region sequences CDR1, CDR2 and CDR3 as shown in SEQ ID Nos: 19, 20 and 21, or amino acid sequence having at least 95% identity to SEQ ID NO: 19, 20 and 21, respectively; or
heavy-chain variable region sequences CDR1, CDR2 and CDR3 as shown in SEQ ID NOs: 7, 8 and 9, or amino acid sequence having at least 95% identity to SEQ ID NO: 7, 8 and 9, respectively, and light-chain variable region sequences CDR1, CDR2 and CDR3 as shown in SEQ ID Nos: 22, 23 and 24, or amino acid sequence having at least 95% identity to SEQ ID NO: 22, 23 and 24, respectively; or
heavy-chain variable region sequences CDR1, CDR2 and CDR3 as shown in SEQ ID Nos: 10, 11 and 12, or amino acid sequence having at least 95% identity to SEQ ID NO: 10, 11 and 12, respectively, and light-chain variable region sequences CDR1, CDR2 and CDR3 as shown in SEQ ID Nos: 25, 26 and 27, or amino acid sequence having at least 95% identity to SEQ ID NO: 25, 26 and 27, respectively; or
heavy-chain variable region sequences CDR1, CDR2 and CDR3 as shown in SEQ ID Nos: 13, 14 and 15, or amino acid sequence having at least 95% identity to SEQ ID NO: 13, 14 and 15, respectively, and light-chain variable region sequences CDR1, CDR2 and CDR3 as shown in SEQ ID Nos: 28, 29 and 30, or amino acid sequence having at least 95% identity to SEQ ID NO: 28, 29 and 30, respectively.

5. The antibody or antigen binding fragment thereof according to claim 1, wherein the antibody or antigen binding fragment thereof specifically recognizes the extracellular region of CD22.

6. The antibody or antigen binding fragment thereof according to claim 1, wherein the antibody comprises at least one of a heavy-chain framework region sequence and a light-chain framework region sequence, at least one part of at least one of the heavy chain framework region sequence and the light chain framework region sequence is derived from at least one of a mouse-derived antibody, a human-derived antibody, a primate-derived antibody or a mutant thereof.

7. The antibody or antigen binding fragment thereof according to claim 1, wherein the antibody has a heavy chain variable region of the amino acid sequence as shown in any one of SEQ ID NOs: 31-35; or
wherein the antibody has a light chain variable region of the amino acid sequence as shown in any one of SEQ ID NOs: 36-40; optionally, the heavy chain variable region of the antibody having the amino acid sequence of SEQ ID NO: 31 and the light chain variable region of the antibody having the amino acid sequence of SEQ ID NO: 36.

8. (canceled)

9. The antibody or antigen binding fragment thereof according to claim 1, wherein the antibody comprises at least one of a heavy-chain constant region and a light-chain constant region, at least one part of at least one of the heavy chain constant region and the light chain constant region is derived from at least one of a mouse-derived antibody, a human-derived antibody, a primate-derived antibody or a mutant thereof:
optionally, the light chain constant region and heavy chain constant region of the antibody are both derived from human-derived IgG antibody or a mutant thereof;
optionally, the light chain constant region and heavy chain constant region of the antibody are both derived from human-derived IgG 1, 2 or 4.

10. (canceled)

11. (canceled)

12. The antibody or antigen binding fragment thereof according to claim 1, wherein the antibody is a single chain antibody, a polymer antibody, a CDR grafted antibody or a micromolecular antibody:
optionally, the antibody is a single chain antibody;
optionally, the single chain antibody has the amino acid sequence as shown in SEQ ID NOs: 41-50;
optionally, the single chain antibody has the amino acid sequence as shown in SEQ ID NO: 41; optionally, the single chain antibody has the amino acid sequence as shown in SEQ ID NO: 46;
optionally, the micromolecular antibody comprises at least one of Fab antibody, Fv antibody, single domain antibody and minimum recognition unit.

13. (canceled)

14. (canceled)

15. (canceled)

16. A nucleic acid molecule, wherein the nucleic acid molecule encodes the antibody or antigen binding fragment thereof of claim 1.

17. The nucleic acid molecule according to claim 16, wherein the nucleic acid molecule is DNA:
optionally, the nucleic acid molecule has a nucleotide sequence as shown in any one of SEQ ID NOs: 51-55 or has a nucleotide sequence as shown in any one of SEQ ID NOs: 56-60 or has a nucleotide sequence as shown in any one of SEQ ID NOs: 61-70.

18. (canceled)

19. An expression vector, wherein it carries the nucleic acid molecule of claim 16;
optionally, the expression vector is an eukaryotic expression vector.

20. (canceled)

21. A recombinant cell, wherein the recombinant cell carries the nucleic acid molecule which encodes the antibody or antigen binding fragment thereof of claim 1, or expresses the antibody or antigen binding fragment thereof of claim 1; optionally, wherein the recombinant cell is obtained by introducing the expression vector which carries the nucleic acid molecule into a host cell; optionally, the recombinant cell is an eukaryotic cell; or the recombinant cell is a mammalian cell.

22. (canceled)

23. (canceled)

24. (canceled)

25. A chimeric antigen receptor, wherein the chimeric antigen receptor comprises:
an extracellular region comprising a heavy chain variable region and a light chain variable region as well as a CD8 hinge region of a single chain antibody, wherein the single chain antibody specifically recognizes CD22;
a transmembrane region comprising an immunocostimulator transmembrane region; and
an intracellular region comprising an intracellular segment of immunocostimulator and CD3 ζ chain;
wherein the heavy chain variable region and light chain variable region of the single chain antibody are the amino acid sequences as defined in claim 1.

26. A CART cell, wherein it expresses the chimeric antigen receptor of claim 25.

27. A pharmaceutical composition, wherein it comprises:
a) the antibody or antigen binding fragment thereof of claim 1,
b) the nucleic acid molecule encoding the antibody or antigen binding fragment thereof of claim 1,
c) the expression vector carrying the nucleic acid molecule which encodes the antibody or antigen binding fragment thereof of claim 1,
d) the recombinant cell carrying the nucleic acid molecule which encodes the antibody or antigen binding fragment thereof of claim 1 or expresses the antibody or antigen binding fragment thereof of claim 1,
e) the chimeric antigen receptor comprising comprises:
an extracellular region comprising a heavy chain variable region and a light chain variable region as well as a CD8 hinge region of a single chain antibody, wherein the single chain antibody specifically recognizes CD22;
a transmembrane region comprising an immunocostimulator transmembrane region; and
an intracellular region comprising an intracellular segment of immunocostimulator and CD3 ζ chain;
wherein the heavy chain variable region and light chain variable region of the single chain antibody are the amino acid sequences as defined in claim 1, or
f) the CART cell expressing the chimeric antigen receptor of e).

28. (canceled)

29. (canceled)

30. (canceled)

31. A kit for detecting CD22, wherein the kit comprises the antibody of claim 1.

32. A method of detecting CD22 or diagnosing CD22 related diseases, wherein the method comprises using the antibody of claim 1 for detecting CD22 or diagnosing CD22 related diseases.

33. A method of treating or preventing a disease, wherein the method comprises administering at least one of the following to a subject having or suspected of having a disease:
A) the antibody or antigen binding fragment thereof of claim 1;
B) the nucleic acid molecule encoding the antibody or antigen binding fragment thereof of claim 1;
C) the expression vector carrying the nucleic acid molecule which encodes the antibody or antigen binding fragment thereof of claim 1;
D) the recombinant cell carrying the nucleic acid molecule which encodes the antibody or antigen binding fragment thereof of claim 1;
E) the chimeric antigen receptor
comprising comprises:
an extracellular region comprising a heavy chain variable region and a light chain variable region as well as a CD8 hinge region of a single chain antibody, wherein the single chain antibody specifically recognizes CD22;
a transmembrane region comprising an immunocostimulator transmembrane region; and
an intracellular region comprising an intracellular segment of immunocostimulator and CD3 chain;
wherein the heavy chain variable region and light chain variable region of the single chain antibody are the amino acid sequences as defined in claim 1;
F) the CART cell expressing the chimeric antigen receptor of E);
G) the pharmaceutical composition comprising at least one of the antibody of claim 1,
wherein, the disease is a CD22-related disease.

34. The method according to claim 33, wherein the CD22-related disease comprises B-lymphocyte leukemia and B-cell lymphoma.

35. (canceled)

36. (canceled)


SEQUENCE LISTING (70 sequences)

SEQ ID NO: 1 - protein, 9 aa, Artificial Sequence
  Synthesized
GYSITSGYY

SEQ ID NO: 2 - protein, 7 aa, Artificial Sequence
  Synthesized
ISYDGSN

SEQ ID NO: 3 - protein, 4 aa, Artificial Sequence
  Synthesized and Xaa is not exist, just for completing the sequence listing.
  misc_feature (3)..(4): Xaa can be any naturally occurring amino acid
TKXX

SEQ ID NO: 4 - protein, 8 aa, Artificial Sequence
  Synthesized
GYNFTSYW

SEQ ID NO: 5 - protein, 8 aa, Artificial Sequence
  Synthesized
IYPGSGNT

SEQ ID NO: 6 - protein, 4 aa, Artificial Sequence
  Synthesized and Xaa is not exist, just for completing the sequence listing.
  misc_feature (3)..(4): Xaa can be any naturally occurring amino acid
ARXX

SEQ ID NO: 7 - protein, 8 aa, Artificial Sequence
  Synthesized
GYTFSSYW

SEQ ID NO: 8 - protein, 8 aa, Artificial Sequence
  Synthesized
ILPGSGST

SEQ ID NO: 9 - protein, 4 aa, Artificial Sequence
  Synthesized and Xaa is not exist, just for completing the sequence listing.
  misc_feature (3)..(4): Xaa can be any naturally occurring amino acid
ARXX

SEQ ID NO: 10 - protein, 8 aa, Artificial Sequence
  Synthesized
GYTFTDSI

SEQ ID NO: 11 - protein, 8 aa, Artificial Sequence
  Synthesized
FYPGSGSI

SEQ ID NO: 12 - protein, 4 aa, Artificial Sequence
  Synthesized
ARHE

SEQ ID NO: 13 - protein, 8 aa, Artificial Sequence
  Synthesized
GYTFSSYW

SEQ ID NO: 14 - protein, 8 aa, Artificial Sequence
  Synthesized
IYPSDSYT

SEQ ID NO: 15 - protein, 4 aa, Artificial Sequence
  Synthesized and Xaa is not exist, just for completing the sequence listing.
  misc_feature (3)..(4): Xaa can be any naturally occurring amino acid
TRXX

SEQ ID NO: 16 - protein, 6 aa, Artificial Sequence
  Synthesized
GNIHNY

SEQ ID NO: 17 - protein, 4 aa, Artificial Sequence
  Synthesized and Xaa is not exist, just for completing the sequence listing.
  misc_feature (4)..(4): Xaa can be any naturally occurring amino acid
NAKX

SEQ ID NO: 18 - protein, 7 aa, Artificial Sequence
  Synthesized
QHFWSTP

SEQ ID NO: 19 - protein, 6 aa, Artificial Sequence
  Synthesized
GNIHNY

SEQ ID NO: 20 - protein, 4 aa, Artificial Sequence
  Synthesized and Xaa is not exist, just for completing the sequence listing.
  misc_feature (4)..(4): Xaa can be any naturally occurring amino acid
NAKX

SEQ ID NO: 21 - protein, 7 aa, Artificial Sequence
  Synthesized
QHFWSTP

SEQ ID NO: 22 - protein, 6 aa, Artificial Sequence
  Synthesized
ENIYSY

SEQ ID NO: 23 - protein, 4 aa, Artificial Sequence
  Synthesized and Xaa is not exist, just for completing the sequence listing.
  misc_feature (4)..(4): Xaa can be any naturally occurring amino acid
NAKX

SEQ ID NO: 24 - protein, 7 aa, Artificial Sequence
  Synthesized
QHHYGSP

SEQ ID NO: 25 - protein, 5 aa, Artificial Sequence
  Synthesized
SSVNY

SEQ ID NO: 26 - protein, 4 aa, Artificial Sequence
  Synthesized and Xaa is not exist, just for completing the sequence listing.
  misc_feature (4)..(4): Xaa can be any naturally occurring amino acid
YTSX

SEQ ID NO: 27 - protein, 7 aa, Artificial Sequence
  Synthesized
QQFTSSP

SEQ ID NO: 28 - protein, 6 aa, Artificial Sequence
  Synthesized
KTISKY

SEQ ID NO: 29 - protein, 4 aa, Artificial Sequence
  Synthesized and Xaa is not exist, just for completing the sequence listing.
  misc_feature (4)..(4): Xaa can be any naturally occurring amino acid
SGSX

SEQ ID NO: 30 - protein, 8 aa, Artificial Sequence
  Synthesized
QQHNEYPW

SEQ ID NO: 31 - protein, 118 aa, Artificial Sequence
  Synthesized
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCTKGGYGYYFDYWGQGTTLTVSS

SEQ ID NO: 32 - protein, 115 aa, Artificial Sequence
  Synthesized
QVQLQQPGAELVKPGTSVKLSCKASGYNFTSYWINWVKLRPGQGLEWIGDIYPGSGNTNYNEKFKSKATLTVDTSSTTAYMQLSSLASEDSALYYCARRGYLDYWGQGTTLTVSS

SEQ ID NO: 33 - protein, 121 aa, Artificial Sequence
  Synthesized
QVQLQQSGAELMKPGASVKISCKATGYTFSSYWIEWVKQRPGHGLEWIGEILPGSGSTNYNEKFKGKATFTADTSSNTAYMQLSSLTSEDSAVYYCARWGQLGLFYAMDYWGQGTSVTVSS

SEQ ID NO: 34 - protein, 119 aa, Artificial Sequence
  Synthesized
KVQLQQSGAGLVKPGASVKLSCKASGYTFTDSILHWLMQRSGQGLEWIGWFYPGSGSIKYNEKFKDKATLTADKSSSTVYMELSRLTSEDSAFYFCARHEDGYDGFAYWGQGTLVTVSA

SEQ ID NO: 35 - protein, 122 aa, Artificial Sequence
  Synthesized
QVQLQQPGAELVRPGASVKLSCKASGYTFSSYWINWVKQRPGQGLEWIGNIYPSDSYTNYNQKFKDKATLTVDKSSSTAYMQLSSPTSEDSAVYYCTREGHYYGSFGAMDYWGQGTSVTVSS

SEQ ID NO: 36 - protein, 108 aa, Artificial Sequence
  Synthesized
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIKR

SEQ ID NO: 37 - protein, 108 aa, Artificial Sequence
  Synthesized
DIQMTQSPASLSASVGETVTITCRASGNIHNYLAWYQQKQGKSPQLLVYNAKTLADGVPSRFSGSGSGTQYSLTINSLQPEDFGSYYCQHFWSTPLTFGAGTKLELKR

SEQ ID NO: 38 - protein, 108 aa, Artificial Sequence
  Synthesized
DIQMTQSPASLSASVGETVTITCRASENIYSYLAWYQQKQGKSPQLLVYNAKTLAEGVPSRFSGSGSGTQFSLKINSLQPEDFGSYYCQHHYGSPLTFGAGTKLELKR

SEQ ID NO: 39 - protein, 107 aa, Artificial Sequence
  Synthesized
ENVLTQSPAIMSASLGEKVTMSCRASSSVNYIFWYQQKSDASPKLWIYYTSNLAPGVPARFSGSGSGNSYSLTISSMEGEDAATYYCQQFTSSPFTFGSGTKLEIKR

SEQ ID NO: 40 - protein, 108 aa, Artificial Sequence
  Synthesized
DVQITQSPSYLAASPGETITINCRASKTISKYLAWYQEKPGKTNKLLIYSGSTLQSGIPSRFSGSGSGTDFTLTISSLEPEDFAMYYCQQHNEYPWTFGGGTKLEIKR

SEQ ID NO: 41 - protein, 241 aa, Artificial Sequence
  Synthesized
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIKRGGGGSGGGGSGGGGSDVQLQESGPGLVKPSQSLSLTCSVTGYSITSGYYWNWIRQFPGNKLEWMGYISYDGSNNYNPSLKNRISITRDTSKNQFFLKLNSVTTEDTATYYCTKGGYGYYFDYWGQGTTLTVSS

SEQ ID NO: 42 - protein, 238 aa, Artificial Sequence
  Synthesized
DIQMTQSPASLSASVGETVTITCRASGNIHNYLAWYQQKQGKSPQLLVYNAKTLADGVPSRFSGSGSGTQYSLTINSLQPEDFGSYYCQHFWSTPLTFGAGTKLELKRGGGGSGGGGSGGGGSQVQLQQPGAELVKPGTSVKLSCKASGYNFTSYWINWVKLRPGQGLEWIGDIYPGSGNTNYNEKFKSKATLTVDTSSTTAYMQLSSLASEDSALYYCARRGYLDYWGQGTTLTVSS

SEQ ID NO: 43 - protein, 244 aa, Artificial Sequence
  Synthesized
DIQMTQSPASLSASVGETVTITCRASENIYSYLAWYQQKQGKSPQLLVYNAKTLAEGVPSRFSGSGSGTQFSLKINSLQPEDFGSYYCQHHYGSPLTFGAGTKLELKRGGGGSGGGGSGGGGSQVQLQQSGAELMKPGASVKISCKATGYTFSSYWIEWVKQRPGHGLEWIGEILPGSGSTNYNEKFKGKATFTADTSSNTAYMQLSSLTSEDSAVYYCARWGQLGLFYAMDYWGQGTSVTVSS

SEQ ID NO: 44 - protein, 241 aa, Artificial Sequence
  Synthesized
ENVLTQSPAIMSASLGEKVTMSCRASSSVNYIFWYQQKSDASPKLWIYYTSNLAPGVPARFSGSGSGNSYSLTISSMEGEDAATYYCQQFTSSPFTFGSGTKLEIKRGGGGSGGGGSGGGGSKVQLQQSGAGLVKPGASVKLSCKASGYTFTDSILHWLMQRSGQGLEWIGWFYPGSGSIKYNEKFKDKATLTADKSSSTVYMELSRLTSEDSAFYFCARHEDGYDGFAYWGQGTLVTVSA

SEQ ID NO: 45 - protein, 245 aa, Artificial Sequence
  Synthesized
DVQITQSPSYLAASPGETITINCRASKTISKYLAWYQEKPGKTNKLLIYSGSTLQSGIPSRFSGSGSGTDFTLTISSLEPEDFAMYYCQQHNEYPWTFGGGTKLEIKRGGGGSGGGGSGGGGSQVQLQQPGAELVRPGASVKLSCKASGYTFSSYWINWVKQRPGQGLEWIGNIYPSDSYTNYNQKFKDKATLTVDKSSSTAYMQLSSPTSEDSAVYYCTREGHYYGSFGAMDYWGQGTSVTVSS

SEQ ID NO: 46 - protein, 241 aa, Artificial Sequence
  Synthesized
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCTKGGYGYYFDYWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIKR

SEQ ID NO: 47 - protein, 238 aa, Artificial Sequence
  Synthesized
QVQLQQPGAELVKPGTSVKLSCKASGYNFTSYWINWVKLRPGQGLEWIGDIYPGSGNTNYNEKFKSKATLTVDTSSTTAYMQLSSLASEDSALYYCARRGYLDYWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLTINSLQPEDFGSYYCQHFWSTPLTFGAGTKLELKR

SEQ ID NO: 48 - protein, 244 aa, Artificial Sequence
  Synthesized
QVQLQQSGAELMKPGASVKISCKATGYTFSSYWIEWVKQRPGHGLEWIGEILPGSGSTNYNEKFKGKATFTADTSSNTAYMQLSSLTSEDSAVYYCARWGQLGLFYAMDYWGQGTSVTVSSGGGGSGGGGSGGGGSDIQMTQSPASLSASVGETVTITCRASENIYSYLAWYQQKQGKSPQLLVYNAKTLAEGVPSRFSGSGSGTQFSLKINSLQPEDFGSYYCQHHYGSPLTFGAGTKLELKR

SEQ ID NO: 49 - protein, 241 aa, Artificial Sequence
  Synthesized
KVQLQQSGAGLVKPGASVKLSCKASGYTFTDSILHWLMQRSGQGLEWIGWFYPGSGSIKYNEKFKDKATLTADKSSSTVYMELSRLTSEDSAFYFCARHEDGYDGFAYWGQGTLVTVSAGGGGSGGGGSGGGGSENVLTQSPAIMSASLGEKVTMSCRASSSVNYIFWYQQKSDASPKLWIYYTSNLAPGVPARFSGSGSGNSYSLTISSMEGEDAATYYCQQFTSSPFTFGSGTKLEIKR

SEQ ID NO: 50 - protein, 245 aa, Artificial Sequence
  Synthesized
QVQLQQPGAELVRPGASVKLSCKASGYTFSSYWINWVKQRPGQGLEWIGNIYPSDSYTNYNQKFKDKATLTVDKSSSTAYMQLSSPTSEDSAVYYCTREGHYYGSFGAMDYWGQGTSVTVSSGGGGSGGGGSGGGGSDVQITQSPSYLAASPGETITINCRASKTISKYLAWYQEKPGKTNKLLIYSGSTLQSGIPSRFSGSGSGTDFTLTISSLEPEDFAMYYCQQHNEYPWTFGGGTKLEIKR

SEQ ID NO: 51 - protein, 354 aa, Artificial Sequence
  Synthesized
GATGTACAGCTTCAGGAGTCAGGACCTGGCCTCGTGAAACCTTCTCAGTCTCTGTCTCTCACCTGCTCTGTCACTGGCTACTCCATCACCAGTGGTTATTACTGGAACTGGATCCGGCAGTTTCCAGGAAACAAACTGGAATGGATGGGCTACATAAGCTACGACGGTAGCAATAACTACAACCCATCTCTCAAAAATCGAATCTCCATCACTCGTGACACATCTAAGAACCAGTTTTTCCTGAAGTTGAATTCTGTGACTACTGAGGACACAGCTACATATTACTGTACAAAAGGGGGCTACGGCTACTACTTTGACTACTGGGGCCAAGGCACCACTCTCACAGTCTCCTCA

SEQ ID NO: 52 - protein, 345 aa, Artificial Sequence
  Synthesized
CAGGTCCAGCTGCAGCAGCCTGGGGCTGAGCTTGTGAAGCCTGGGACTTCAGTGAAGCTGTCCTGCAAGGCTTCTGGCTACAACTTCACCAGCTACTGGATAAACTGGGTGAAGCTGAGGCCTGGACAAGGCCTTGAGTGGATTGGAGATATTTATCCTGGTAGTGGTAATACTAATTACAATGAGAAGTTCAAGAGCAAGGCCACACTGACTGTAGACACATCCTCCACCACAGCCTACATGCAACTTAGTAGCCTGGCCTCTGAGGACTCTGCTCTCTATTACTGTGCAAGACGGGGGTATCTTGACTACTGGGGCCAAGGCACCACTCTCACAGTCTCCTCA

SEQ ID NO: 53 - protein, 363 aa, Artificial Sequence
  Synthesized
CAGGTTCAGCTGCAGCAGTCTGGAGCTGAGCTGATGAAGCCTGGGGCCTCAGTGAAGATATCCTGCAAGGCTACTGGCTACACATTCAGTAGCTACTGGATAGAGTGGGTAAAGCAGAGGCCTGGACATGGCCTTGAGTGGATTGGAGAGATTTTACCTGGAAGTGGTAGTACTAACTACAATGAGAAGTTCAAGGGCAAGGCCACATTCACTGCAGATACATCCTCCAACACAGCCTACATGCAACTCAGCAGCCTGACATCTGAGGACTCTGCCGTCTATTACTGTGCAAGATGGGGGCAGCTCGGGCTTTTTTATGCTATGGACTACTGGGGTCAAGGAACCTCAGTCACCGTCTCCTCA

SEQ ID NO: 54 - protein, 357 aa, Artificial Sequence
  Synthesized
AAGGTCCAGCTGCAGCAGTCTGGAGCTGGGCTGGTGAAACCCGGGGCATCAGTGAAGCTGTCCTGCAAGGCTTCTGGCTACACCTTCACTGACTCTATTTTACACTGGCTAATGCAGAGATCTGGACAGGGTCTTGAGTGGATTGGGTGGTTTTACCCTGGAAGTGGTAGTATAAAGTACAATGAGAAATTCAAGGACAAGGCCACATTGACTGCGGACAAGTCCTCCAGCACAGTCTATATGGAGCTTAGTAGATTGACATCTGAAGACTCTGCGTTCTATTTCTGTGCAAGGCACGAAGATGGTTACGACGGGTTTGCTTACTGGGGCCAAGGGACTCTGGTCACTGTCTCTGCA

SEQ ID NO: 55 - protein, 366 aa, Artificial Sequence
  Synthesized
CAGGTCCAACTGCAGCAGCCTGGGGCTGAGCTGGTGAGGCCTGGGGCTTCAGTGAAGCTGTCCTGCAAGGCTTCTGGCTACACCTTCAGCAGCTACTGGATAAACTGGGTGAAGCAGAGGCCTGGACAAGGCCTTGAGTGGATCGGAAATATTTATCCTTCTGATAGTTATACTAACTACAATCAAAAGTTCAAGGACAAGGCCACATTGACTGTAGACAAATCCTCCAGTACAGCCTACATGCAGCTCAGCAGCCCGACATCTGAGGACTCTGCGGTCTATTACTGTACAAGAGAGGGGCATTACTACGGATCCTTCGGTGCTATGGACTACTGGGGTCAAGGAACCTCAGTCACCGTCTCCTCA

SEQ ID NO: 56 - protein, 324 aa, Artificial Sequence
  Synthesized
GACATCCAGATGACTCAGTCTCCAGCCTCCCTATCTGCATCTGTGGGAGAAACTGTCACCATCACATGTCGAGCAAGTGGGAATATTCACAATTATTTAGCATGGTATCAGCAGAAACAGGGAAAATCTCCTCAGCTCCTGGTCTATAATGCAAAAACCTTAGCAGATGGTGTGCCATCAAGGTTCAGTGGCAGTGGATCAGGAACACAATATTCTCTCAAGATCAACAGCCTGCAGCCTGAAGATTTTGGGAGTTATTACTGTCAACATTTTTGGAGTACTCCTCCGACGTTCGGTGGAGGCACCAAGCTGGAAATCAAACGG

SEQ ID NO: 57 - protein, 324 aa, Artificial Sequence
  Synthesized
GACATCCAGATGACTCAGTCTCCAGCCTCCCTATCTGCATCTGTGGGAGAAACTGTCACCATCACATGTCGAGCAAGTGGGAATATTCACAATTATTTAGCATGGTATCAGCAGAAACAGGGAAAATCTCCTCAGCTCCTGGTCTATAATGCAAAAACCTTAGCAGATGGTGTGCCATCAAGGTTCAGTGGCAGTGGATCAGGAACACAATATTCTCTCACGATCAACAGCCTGCAGCCTGAAGATTTTGGGAGTTACTACTGTCAACATTTTTGGAGTACTCCTCTCACGTTCGGTGCTGGGACCAAGCTGGAGCTGAAACGG

SEQ ID NO: 58 - protein, 324 aa, Artificial Sequence
  Synthesized
GACATCCAGATGACTCAGTCTCCAGCCTCCCTATCTGCATCTGTGGGAGAAACTGTCACCATCACATGTCGAGCAAGTGAGAATATTTACAGTTATTTAGCATGGTATCAGCAGAAACAGGGAAAATCTCCTCAGCTCCTGGTCTATAATGCAAAAACCTTAGCAGAAGGTGTGCCATCAAGGTTCAGTGGCAGTGGATCAGGCACACAGTTTTCTCTGAAGATCAACAGCCTGCAGCCTGAAGATTTTGGGAGTTATTACTGTCAACATCATTATGGTAGTCCGCTCACGTTCGGTGCTGGGACCAAGCTGGAGCTGAAACGG

SEQ ID NO: 59 - protein, 321 aa, Artificial Sequence
  Synthesized
GAAAATGTGCTCACCCAGTCTCCAGCAATCATGTCTGCATCTCTAGGGGAGAAGGTCACCATGAGCTGCAGGGCCAGCTCAAGTGTAAATTACATATTCTGGTACCAGCAGAAGTCAGATGCCTCCCCCAAACTATGGATTTATTACACATCCAACCTGGCTCCTGGAGTCCCAGCTCGCTTCAGTGGCAGTGGGTCTGGGAACTCTTATTCTCTCACAATCAGCAGCATGGAGGGTGAAGATGCTGCCACTTATTACTGCCAGCAGTTTACTAGTTCCCCATTCACGTTCGGCTCGGGGACAAAGTTGGAAATAAAACGG

SEQ ID NO: 60 - protein, 324 aa, Artificial Sequence
  Synthesized
GATGTCCAGATAACCCAGTCTCCATCTTATCTTGCTGCATCTCCTGGAGAAACCATTACTATTAATTGCAGGGCAAGTAAGACCATTAGCAAATATTTGGCCTGGTATCAAGAGAAACCTGGGAAAACTAATAAGCTTCTTATCTACTCTGGATCCACTTTGCAATCTGGAATTCCATCAAGGTTCAGTGGCAGTGGATCTGGTACAGATTTCACTCTCACCATCAGTAGCCTGGAGCCTGAAGATTTTGCAATGTATTACTGTCAACAACATAATGAATACCCGTGGACGTTCGGTGGAGGCACCAAGCTGGAAATCAAACGG

SEQ ID NO: 61 - protein, 723 aa, Artificial Sequence
  Synthesized
GACATCCAGATGACTCAGTCTCCAGCCTCCCTATCTGCATCTGTGGGAGAAACTGTCACCATCACATGTCGAGCAAGTGGGAATATTCACAATTATTTAGCATGGTATCAGCAGAAACAGGGAAAATCTCCTCAGCTCCTGGTCTATAATGCAAAAACCTTAGCAGATGGTGTGCCATCAAGGTTCAGTGGCAGTGGATCAGGAACACAATATTCTCTCAAGATCAACAGCCTGCAGCCTGAAGATTTTGGGAGTTATTACTGTCAACATTTTTGGAGTACTCCTCCGACGTTCGGTGGAGGCACCAAGCTGGAAATCAAACGGGGAGGAGGAGGCTCCGGCGGAGGAGGCTCTGGAGGAGGAGGCAGCGATGTACAGCTTCAGGAGTCAGGACCTGGCCTCGTGAAACCTTCTCAGTCTCTGTCTCTCACCTGCTCTGTCACTGGCTACTCCATCACCAGTGGTTATTACTGGAACTGGATCCGGCAGTTTCCAGGAAACAAACTGGAATGGATGGGCTACATAAGCTACGACGGTAGCAATAACTACAACCCATCTCTCAAAAATCGAATCTCCATCACTCGTGACACATCTAAGAACCAGTTTTTCCTGAAGTTGAATTCTGTGACTACTGAGGACACAGCTACATATTACTGTACAAAAGGGGGCTACGGCTACTACTTTGACTACTGGGGCCAAGGCACCACTCTCACAGTCTCCTCA

SEQ ID NO: 62 - protein, 714 aa, Artificial Sequence
  Synthesized
GACATCCAGATGACTCAGTCTCCAGCCTCCCTATCTGCATCTGTGGGAGAAACTGTCACCATCACATGTCGAGCAAGTGGGAATATTCACAATTATTTAGCATGGTATCAGCAGAAACAGGGAAAATCTCCTCAGCTCCTGGTCTATAATGCAAAAACCTTAGCAGATGGTGTGCCATCAAGGTTCAGTGGCAGTGGATCAGGAACACAATATTCTCTCACGATCAACAGCCTGCAGCCTGAAGATTTTGGGAGTTACTACTGTCAACATTTTTGGAGTACTCCTCTCACGTTCGGTGCTGGGACCAAGCTGGAGCTGAAACGGGGAGGAGGAGGCTCCGGCGGAGGAGGCTCTGGAGGAGGAGGCAGCCAGGTCCAGCTGCAGCAGCCTGGGGCTGAGCTTGTGAAGCCTGGGACTTCAGTGAAGCTGTCCTGCAAGGCTTCTGGCTACAACTTCACCAGCTACTGGATAAACTGGGTGAAGCTGAGGCCTGGACAAGGCCTTGAGTGGATTGGAGATATTTATCCTGGTAGTGGTAATACTAATTACAATGAGAAGTTCAAGAGCAAGGCCACACTGACTGTAGACACATCCTCCACCACAGCCTACATGCAACTTAGTAGCCTGGCCTCTGAGGACTCTGCTCTCTATTACTGTGCAAGACGGGGGTATCTTGACTACTGGGGCCAAGGCACCACTCTCACAGTCTCCTCA

SEQ ID NO: 63 - protein, 732 aa, Artificial Sequence
  Synthesized
GACATCCAGATGACTCAGTCTCCAGCCTCCCTATCTGCATCTGTGGGAGAAACTGTCACCATCACATGTCGAGCAAGTGAGAATATTTACAGTTATTTAGCATGGTATCAGCAGAAACAGGGAAAATCTCCTCAGCTCCTGGTCTATAATGCAAAAACCTTAGCAGAAGGTGTGCCATCAAGGTTCAGTGGCAGTGGATCAGGCACACAGTTTTCTCTGAAGATCAACAGCCTGCAGCCTGAAGATTTTGGGAGTTATTACTGTCAACATCATTATGGTAGTCCGCTCACGTTCGGTGCTGGGACCAAGCTGGAGCTGAAACGGGGAGGAGGAGGCTCCGGCGGAGGAGGCTCTGGAGGAGGAGGCAGCCAGGTTCAGCTGCAGCAGTCTGGAGCTGAGCTGATGAAGCCTGGGGCCTCAGTGAAGATATCCTGCAAGGCTACTGGCTACACATTCAGTAGCTACTGGATAGAGTGGGTAAAGCAGAGGCCTGGACATGGCCTTGAGTGGATTGGAGAGATTTTACCTGGAAGTGGTAGTACTAACTACAATGAGAAGTTCAAGGGCAAGGCCACATTCACTGCAGATACATCCTCCAACACAGCCTACATGCAACTCAGCAGCCTGACATCTGAGGACTCTGCCGTCTATTACTGTGCAAGATGGGGGCAGCTCGGGCTTTTTTATGCTATGGACTACTGGGGTCAAGGAACCTCAGTCACCGTCTCCTCA

SEQ ID NO: 64 - protein, 723 aa, Artificial Sequence
  Synthesized
GAAAATGTGCTCACCCAGTCTCCAGCAATCATGTCTGCATCTCTAGGGGAGAAGGTCACCATGAGCTGCAGGGCCAGCTCAAGTGTAAATTACATATTCTGGTACCAGCAGAAGTCAGATGCCTCCCCCAAACTATGGATTTATTACACATCCAACCTGGCTCCTGGAGTCCCAGCTCGCTTCAGTGGCAGTGGGTCTGGGAACTCTTATTCTCTCACAATCAGCAGCATGGAGGGTGAAGATGCTGCCACTTATTACTGCCAGCAGTTTACTAGTTCCCCATTCACGTTCGGCTCGGGGACAAAGTTGGAAATAAAACGGGGAGGAGGAGGCTCCGGCGGAGGAGGCTCTGGAGGAGGAGGCAGCAAGGTCCAGCTGCAGCAGTCTGGAGCTGGGCTGGTGAAACCCGGGGCATCAGTGAAGCTGTCCTGCAAGGCTTCTGGCTACACCTTCACTGACTCTATTTTACACTGGCTAATGCAGAGATCTGGACAGGGTCTTGAGTGGATTGGGTGGTTTTACCCTGGAAGTGGTAGTATAAAGTACAATGAGAAATTCAAGGACAAGGCCACATTGACTGCGGACAAGTCCTCCAGCACAGTCTATATGGAGCTTAGTAGATTGACATCTGAAGACTCTGCGTTCTATTTCTGTGCAAGGCACGAAGATGGTTACGACGGGTTTGCTTACTGGGGCCAAGGGACTCTGGTCACTGTCTCTGCA

SEQ ID NO: 65 - protein, 735 aa, Artificial Sequence
  Synthesized
GATGTCCAGATAACCCAGTCTCCATCTTATCTTGCTGCATCTCCTGGAGAAACCATTACTATTAATTGCAGGGCAAGTAAGACCATTAGCAAATATTTGGCCTGGTATCAAGAGAAACCTGGGAAAACTAATAAGCTTCTTATCTACTCTGGATCCACTTTGCAATCTGGAATTCCATCAAGGTTCAGTGGCAGTGGATCTGGTACAGATTTCACTCTCACCATCAGTAGCCTGGAGCCTGAAGATTTTGCAATGTATTACTGTCAACAACATAATGAATACCCGTGGACGTTCGGTGGAGGCACCAAGCTGGAAATCAAACGGGGAGGAGGAGGCTCCGGCGGAGGAGGCTCTGGAGGAGGAGGCAGCCAGGTCCAACTGCAGCAGCCTGGGGCTGAGCTGGTGAGGCCTGGGGCTTCAGTGAAGCTGTCCTGCAAGGCTTCTGGCTACACCTTCAGCAGCTACTGGATAAACTGGGTGAAGCAGAGGCCTGGACAAGGCCTTGAGTGGATCGGAAATATTTATCCTTCTGATAGTTATACTAACTACAATCAAAAGTTCAAGGACAAGGCCACATTGACTGTAGACAAATCCTCCAGTACAGCCTACATGCAGCTCAGCAGCCCGACATCTGAGGACTCTGCGGTCTATTACTGTACAAGAGAGGGGCATTACTACGGATCCTTCGGTGCTATGGACTACTGGGGTCAAGGAACCTCAGTCACCGTCTCCTCA

SEQ ID NO: 66 - protein, 723 aa, Artificial Sequence
  Synthesized
GATGTACAGCTTCAGGAGTCAGGACCTGGCCTCGTGAAACCTTCTCAGTCTCTGTCTCTCACCTGCTCTGTCACTGGCTACTCCATCACCAGTGGTTATTACTGGAACTGGATCCGGCAGTTTCCAGGAAACAAACTGGAATGGATGGGCTACATAAGCTACGACGGTAGCAATAACTACAACCCATCTCTCAAAAATCGAATCTCCATCACTCGTGACACATCTAAGAACCAGTTTTTCCTGAAGTTGAATTCTGTGACTACTGAGGACACAGCTACATATTACTGTACAAAAGGGGGCTACGGCTACTACTTTGACTACTGGGGCCAAGGCACCACTCTCACAGTCTCCTCAGGAGGAGGAGGCTCCGGCGGAGGAGGCTCTGGAGGAGGAGGCAGCGACATCCAGATGACTCAGTCTCCAGCCTCCCTATCTGCATCTGTGGGAGAAACTGTCACCATCACATGTCGAGCAAGTGGGAATATTCACAATTATTTAGCATGGTATCAGCAGAAACAGGGAAAATCTCCTCAGCTCCTGGTCTATAATGCAAAAACCTTAGCAGATGGTGTGCCATCAAGGTTCAGTGGCAGTGGATCAGGAACACAATATTCTCTCAAGATCAACAGCCTGCAGCCTGAAGATTTTGGGAGTTATTACTGTCAACATTTTTGGAGTACTCCTCCGACGTTCGGTGGAGGCACCAAGCTGGAAATCAAACGG

SEQ ID NO: 67 - protein, 714 aa, Artificial Sequence
  Synthesized
CAGGTCCAGCTGCAGCAGCCTGGGGCTGAGCTTGTGAAGCCTGGGACTTCAGTGAAGCTGTCCTGCAAGGCTTCTGGCTACAACTTCACCAGCTACTGGATAAACTGGGTGAAGCTGAGGCCTGGACAAGGCCTTGAGTGGATTGGAGATATTTATCCTGGTAGTGGTAATACTAATTACAATGAGAAGTTCAAGAGCAAGGCCACACTGACTGTAGACACATCCTCCACCACAGCCTACATGCAACTTAGTAGCCTGGCCTCTGAGGACTCTGCTCTCTATTACTGTGCAAGACGGGGGTATCTTGACTACTGGGGCCAAGGCACCACTCTCACAGTCTCCTCAGGAGGAGGAGGCTCCGGCGGAGGAGGCTCTGGAGGAGGAGGCAGCGACATCCAGATGACTCAGTCTCCAGCCTCCCTATCTGCATCTGTGGGAGAAACTGTCACCATCACATGTCGAGCAAGTGGGAATATTCACAATTATTTAGCATGGTATCAGCAGAAACAGGGAAAATCTCCTCAGCTCCTGGTCTATAATGCAAAAACCTTAGCAGATGGTGTGCCATCAAGGTTCAGTGGCAGTGGATCAGGAACACAATATTCTCTCACGATCAACAGCCTGCAGCCTGAAGATTTTGGGAGTTACTACTGTCAACATTTTTGGAGTACTCCTCTCACGTTCGGTGCTGGGACCAAGCTGGAGCTGAAACGG

SEQ ID NO: 68 - protein, 732 aa, Artificial Sequence
  Synthesized
CAGGTTCAGCTGCAGCAGTCTGGAGCTGAGCTGATGAAGCCTGGGGCCTCAGTGAAGATATCCTGCAAGGCTACTGGCTACACATTCAGTAGCTACTGGATAGAGTGGGTAAAGCAGAGGCCTGGACATGGCCTTGAGTGGATTGGAGAGATTTTACCTGGAAGTGGTAGTACTAACTACAATGAGAAGTTCAAGGGCAAGGCCACATTCACTGCAGATACATCCTCCAACACAGCCTACATGCAACTCAGCAGCCTGACATCTGAGGACTCTGCCGTCTATTACTGTGCAAGATGGGGGCAGCTCGGGCTTTTTTATGCTATGGACTACTGGGGTCAAGGAACCTCAGTCACCGTCTCCTCAGGAGGAGGAGGCTCCGGCGGAGGAGGCTCTGGAGGAGGAGGCAGCGACATCCAGATGACTCAGTCTCCAGCCTCCCTATCTGCATCTGTGGGAGAAACTGTCACCATCACATGTCGAGCAAGTGAGAATATTTACAGTTATTTAGCATGGTATCAGCAGAAACAGGGAAAATCTCCTCAGCTCCTGGTCTATAATGCAAAAACCTTAGCAGAAGGTGTGCCATCAAGGTTCAGTGGCAGTGGATCAGGCACACAGTTTTCTCTGAAGATCAACAGCCTGCAGCCTGAAGATTTTGGGAGTTATTACTGTCAACATCATTATGGTAGTCCGCTCACGTTCGGTGCTGGGACCAAGCTGGAGCTGAAACGG

SEQ ID NO: 69 - protein, 723 aa, Artificial Sequence
  Synthesized
AAGGTCCAGCTGCAGCAGTCTGGAGCTGGGCTGGTGAAACCCGGGGCATCAGTGAAGCTGTCCTGCAAGGCTTCTGGCTACACCTTCACTGACTCTATTTTACACTGGCTAATGCAGAGATCTGGACAGGGTCTTGAGTGGATTGGGTGGTTTTACCCTGGAAGTGGTAGTATAAAGTACAATGAGAAATTCAAGGACAAGGCCACATTGACTGCGGACAAGTCCTCCAGCACAGTCTATATGGAGCTTAGTAGATTGACATCTGAAGACTCTGCGTTCTATTTCTGTGCAAGGCACGAAGATGGTTACGACGGGTTTGCTTACTGGGGCCAAGGGACTCTGGTCACTGTCTCTGCAGGAGGAGGAGGCTCCGGCGGAGGAGGCTCTGGAGGAGGAGGCAGCGAAAATGTGCTCACCCAGTCTCCAGCAATCATGTCTGCATCTCTAGGGGAGAAGGTCACCATGAGCTGCAGGGCCAGCTCAAGTGTAAATTACATATTCTGGTACCAGCAGAAGTCAGATGCCTCCCCCAAACTATGGATTTATTACACATCCAACCTGGCTCCTGGAGTCCCAGCTCGCTTCAGTGGCAGTGGGTCTGGGAACTCTTATTCTCTCACAATCAGCAGCATGGAGGGTGAAGATGCTGCCACTTATTACTGCCAGCAGTTTACTAGTTCCCCATTCACGTTCGGCTCGGGGACAAAGTTGGAAATAAAACGG

SEQ ID NO: 70 - protein, 735 aa, Artificial Sequence
  Synthesized
CAGGTCCAACTGCAGCAGCCTGGGGCTGAGCTGGTGAGGCCTGGGGCTTCAGTGAAGCTGTCCTGCAAGGCTTCTGGCTACACCTTCAGCAGCTACTGGATAAACTGGGTGAAGCAGAGGCCTGGACAAGGCCTTGAGTGGATCGGAAATATTTATCCTTCTGATAGTTATACTAACTACAATCAAAAGTTCAAGGACAAGGCCACATTGACTGTAGACAAATCCTCCAGTACAGCCTACATGCAGCTCAGCAGCCCGACATCTGAGGACTCTGCGGTCTATTACTGTACAAGAGAGGGGCATTACTACGGATCCTTCGGTGCTATGGACTACTGGGGTCAAGGAACCTCAGTCACCGTCTCCTCAGGAGGAGGAGGCTCCGGCGGAGGAGGCTCTGGAGGAGGAGGCAGCGATGTCCAGATAACCCAGTCTCCATCTTATCTTGCTGCATCTCCTGGAGAAACCATTACTATTAATTGCAGGGCAAGTAAGACCATTAGCAAATATTTGGCCTGGTATCAAGAGAAACCTGGGAAAACTAATAAGCTTCTTATCTACTCTGGATCCACTTTGCAATCTGGAATTCCATCAAGGTTCAGTGGCAGTGGATCTGGTACAGATTTCACTCTCACCATCAGTAGCCTGGAGCCTGAAGATTTTGCAATGTATTACTGTCAACAACATAATGAATACCCGTGGACGTTCGGTGGAGGCACCAAGCTGGAAATCAAACGG